(19) United States
(12) Patent Application Publication
(10) Pub. No.: US 2022/0120753 A1
(43) Pub. Date: Apr. 21, 2022

(54) METHOD FOR DETERMINING PROGNOSIS OF CANCER

(71) Applicants: The University of Tokyo, Tokyo (JP); St. Marianna University School of Medicine, Kanagawa (JP)

(72) Inventors: Makoto NAKANISHI, Tokyo (JP); Yoshikazu JOHMURA, Tokyo (JP); Tomohiko OHTA, Kanagawa (JP)

(73) Assignees: The University of Tokyo, Tokyo (JP); St. Marianna University School of Medicine, Kanagawa (JP)

(21) Appl. No.: 17/276,951

(22) PCT Filed: Sep. 20, 2019
(86) PCT No.: PCT/JP2019/036949

§ 371 Date: Dec. 13, 2021

(30) Foreign Application Priority Data
Sep. 21, 2018 (JP) 2018-177864

(51) Int. Cl. G01N 33/574; A61K 31/138; A61P 35/00

(52) U.S. Cl. CPC G01N 33/57484; G01N 2474/20; A61P 35/00; A61K 31/138

(57) ABSTRACT

The present invention is intended to provide a method for determining the prognosis of a hormone receptor-positive cancer (for example, an ERα-positive breast cancer, an ERα-positive endometrial cancer, etc.) or a method for assisting the determination of the prognosis, and a method for evaluating the sensitivity of the cancer to antihormone therapy or a method for assisting the evaluation of the sensitivity of the cancer to antihormone therapy. Specifically, the present invention relates to a method for determining the prognosis of a hormone receptor-positive cancer, or a method for assisting the determination of the prognosis, which comprises the following steps (a) and (b): (a) a step of detecting Fbxo22-positive cancer cells in a sample derived from the cancer tissues; and (b) a step of calculating the percentage of the Fbxo22-positive cancer cells to the cells present in the sample.

### TECHNICAL FIELD

The present invention relates to a method for determining the prognosis of a cancer, in particular, a hormone receptor-positive cancer, and a method for determining the sensitivity of a hormone receptor-positive cancer to antihormone therapy.

### BACKGROUND ART

Hormone therapy is a representative method for treating a hormone receptor-positive cancer that proliferates due to stimulation by estrogen or androgen, and examples of such hormone therapy may include antiestrogen therapy to breast cancer and antiandrogen therapy to prostate cancer.

Estrogen receptor α (ERα) belongs to the nuclear receptor superfamily, and the estrogen receptor α functions as a ligand-activated transcription factor that regulates initiation of transcription of various target genes located downstream thereof, when estrogen binds to the ERα. The transcriptional regulation function of the estrogen receptor is provoked as a result of the formation of a complex with various coactivators or corepressors, and thereby, a change in the chromatin structure of the promoter region of the target gene is induced.

Approximately 70% of breast cancers are ERα positive. Tamoxifen (TAM) is a standard therapeutic drug to patients with ERα-positive breast cancer, in particular, premenopausal patients with ERα-positive breast cancer (Non Patent Literature 1 and Non Patent Literature 2). It has been reported that the annual breast cancer mortality rate is reduced by approximately 30% according to TAM therapy (Non Patent Literature 3 and Non Patent Literature 4). However, it has also been reported that approximately 25% of early breast cancer patients treated by TAM have a recurrence within 15 years (Non Patent Literature 5). Hence, it is considered that a certain percentage of breast cancer patients need to be reviewed regarding the therapeutic method therefor. How to specify such patients who need to be reviewed regarding the therapeutic method therefor has become an important issue.

TAM is one type of selective estrogen receptor modulator (SERM). That is, TAM functions as with estrogen in certain tissues, but in other tissues, TAM inhibits the function of estrogen, like selective estrogen receptor degraders (SERDs) such as Fulvestrant (Non Patent Literature 6 and Non Patent Literature 7). According to previous studies, it has been elucidated that the relative expression of a coactivator and a corepressor in a cell contributes to the selective regulation of ERα function (Non Patent Literature 8 to Non Patent Literature 11). However, since certain types of ERα-positive/HER2-negative breast cancers exhibit TAM resistance, it is considered that there is an unclarified phenomenon in the molecular dynamics of SERM and a cofactor that cooperates with the SERM.

The KDM4 family of histone lysine demethylase induces a chromatin structural change in response to steroid hormone, and plays an important role in transcriptional regulation. For instance, KDM4C is a coactivator essential for transcription via an androgen receptor and androgen-dependent cell growth in prostate cancer (Non Patent Literature 12 and Non Patent Literature 13). Moreover, it has been reported that KDM4B is associated with ERα-inducible transcription and cell growth in breast cancer cells, through direct interaction with ERα and the lysine demethylation of KDM4B (Non Patent Literature 14 to Non Patent Literature 17).

There are many unclear points regarding the function of Fbxo22 (F-box 22 protein). Fbxo22 is an F-box protein consisting of three functional domains (F-box, FIST-N and FIST-C). Fbxo22 has been reported as a transcriptional target of p53 (Non Patent Literature 18), and thereafter, it has been clarified that Fbxo22 forms a complex with KDM4A (Non Patent Literature 19). The present inventors have identified that an SCF^{Fbxo22}-KDM4A complex functions as E3 ubiquitin ligase that targets methylated p53, and is an essential constituent in aging (Non Patent Literature 20). However, to date, only a part of the SCF^{Fbxo22} targets has been elucidated.

As mentioned above, when SERM such as TAM functions as an antihormone therapeutic agent for cancers, many coactivators and corepressors are associated with the antihormone therapeutic agent. It is considered that elucidation of the molecular dynamics of these cofactors leads to clarification of the action mechanism of SERM, which is used to investigate a cause of the TAM resistance of breast cancer. Nevertheless, the molecular dynamics of the above-described cofactors have not yet been completely clarified at the present moment.

### CITATION LIST

### Non Patent Literature

Non Patent Literature 1: Osborne et al., N Engl J Med. 339: 1609-1618, 1998
Non Patent Literature 2: Coates et al., Ann Oncol. 26: 1533-1546, 2015
Non Patent Literature 3: Group EBCTC. Lancet. 365: 1687-1717, 2005
Non Patent Literature 4: Davies et al., Lancet. 378: 771-784, 2011
Non Patent Literature 5: Davies et al., Lancet. 381: 805-816, 2013
Non Patent Literature 6: Nettles and Greene Annual review of physiology. 67: 309-333, 2005
Non Patent Literature 7: Smith and O'Malley Endocrine reviews. 25: 45-71, 2004
Non Patent Literature 8: Shang et al., Science (New York, N.Y.). 295: 2465-2468, 2002
Non Patent Literature 9: Smith et al., Molecular endocrinology (Baltimore, Md.). 11: 657-666, 1997
Non Patent Literature 10: Keeton and Brown Molecular endocrinology (Baltimore, Md.). 19: 1543-1554, 2005
Non Patent Literature 11: Girault et al., Cancer Research. 9: 1259-1266, 2003
Non Patent Literature 12: Wissmann et al., Nat Cell Biol. 9: 347-53, 2007
Non Patent Literature 13: Crea et al., Mol Cancer. 11: 52, 2012
Non Patent Literature 14: Yang et al., Cancer Res. 70: 6456-66, 2010
Non Patent Literature 15: Shi et al., Proc Natl Acad Sci USA. 108: 7541-7546, 2011
Non Patent Literature 16: Kawazu et al., PLoS One. 6: e17830, 2011
Non Patent Literature 17: Gaughan et al., Nucleic Acids Res. 41: 6892-6904, 2013
Non Patent Literature 18: Vrba et al., BMC Genomics. 9: 486, 2008
Non Patent Literature 19: Tan et al., Mol Cell Biol. 31: 3687-3699, 2011
Non Patent Literature 20: Johmura et al., Nat Commun. 7: 10574, 2016

### SUMMARY OF INVENTION

### Technical Problem

Under the aforementioned circumstances, according to the present inventors, it is an object of the present invention to provide a method for determining the prognosis of a hormone receptor-positive cancer (for example, an ERα-positive breast cancer, an ERα-positive endometrial cancer, etc.) or a method for assisting the determination of the prognosis (for example, a method of providing information necessary for determination of the prognosis, etc.), and a method for evaluating sensitivity to a therapy using an antihormone therapeutic agent (for example, SERM, etc.) or a method for assisting the evaluation of the therapy sensitivity (for example, a method of providing information necessary for the evaluation of the therapy sensitivity, etc.).

### Solution to Problem

The present inventors have conducted intensive studies directed towards elucidating the molecular basis of a mechanism of controlling the sensitivity of ERα-positive breast cancer cells to TAM. Aa a result, the inventors have clarified the following points.

First, the present inventors have revealed that the function of one of SERMs, TAM, as an antagonist against ERα is induced by the selective decomposition of KDM4B forming a complex with TAM-bound ERα by an SCF-Fbxo22 complex (SCF^{Fbxo22}) (see FIG. 1), and have demonstrated that if the expression level of Fbxo22 is low in an ERα-positive cancer, the cancer is resistant to the antihormone therapy with SERM.

Moreover, the present inventors have revealed that if the expression level of Fbxo22 is low in an ERα-positive breast cancer, the cancer has a poor prognosis, and have demonstrated that Fbxo22 is a novel excellent prognosis-determining factor, which is independent from known prognosis-determining factors (tumor grade, lymph node metastasis, progesterone receptor, the expression level of Ki67, etc.).

The present invention has been completed based on the aforementioned findings.

Specifically, the present invention includes the following (1) to (15).

(1) A method for determining the prognosis of a hormone receptor-positive cancer, or a method for assisting the determination of the prognosis, which comprises the following steps (a) and (b):

(a) a step of detecting Fbxo22-positive cancer cells in a sample derived from the cancer tissues; and

(b) a step of calculating the percentage of the Fbxo22-positive cancer cells to cancer cells present in the sample.

(2) The method according to the above (1), which is characterized in that the cancer is determined to have a poor prognosis, when the percentage of the Fbxo22-positive cancer cells is less than 10%.
(3) The method according to the above (1) or (2), which is characterized in that the expression of the Fbxo22 is detected by an immunohistochemical staining method.
(4) The method according to the above (3), which is characterized in that the Fbxo22-positive cancer cells are evaluated based on the stainability of the cells by an anti-Fbxo22 antibody.
(5) The method according to the above (4), which is characterized in that the cells are evaluated to be Fbxo22-positive cancer cells, when the stainability is a moderate level or higher.
(6) The method according to any one of the above (1) to (5), which is characterized in that the hormone receptor-positive cancer is either an ERα (estrogen receptor α)-positive cancer or an AR (androgen receptor)-positive cancer.
(7) The method according to the above (6), which is characterized in that the ERα-positive cancer is an ERα-positive breast cancer or an ERα-positive endometrial cancer.
(8) The method according to the above (6), which is characterized in that the AR-positive cancer is an AR-positive prostate cancer.
(9) A method for evaluating the therapeutic effects of an anti-hormonal agent on a hormone receptor-positive cancer based on the results obtained by the method according to any one of the above (1) to (5), or a method for supplementally evaluating the therapeutic effects.
(10) The method according to the above (9), which is characterized in that the hormone receptor-positive cancer is either an ERα-positive cancer or an AR-positive cancer.
(11) The method according to the above (10), which is characterized in that the ERα-positive cancer is an ERα-positive breast cancer or an ERα-positive endometrial cancer.
(12) The method according to the above (11), which is characterized in that the anti-hormonal agent is SERM (selective estrogen receptor modulator).
(13) The method according to the above (10), which is characterized in that the AR-positive cancer is an AR-positive prostate cancer.
(14) The method according to the above (13), which is characterized in that the anti-hormonal agent is SARM (selective androgen receptor modulator).
(15) A kit for determining the prognosis of a hormone receptor-positive cancer, or for evaluating the therapeutic effects of an anti-hormonal agent on the cancer, wherein the kit comprises an element for detecting the expression level of Fbxo22.

### Advantageous Effects of Invention

The method for determining prognosis and the method for assisting the determination of the prognosis of the present invention are methods of using a prognostic factor that is independent from conventional prognostic factors, and in particular, the present methods exhibit excellent effects on the diagnosis of the prognosis of a poor prognostic Luminal A breast cancer, the diagnosis of which is generally impossible.

According to the method for evaluating therapy sensitivity and the method for supplementally evaluating therapy sensitivity of the present invention, the therapeutic effects of an antihormone therapeutic agent (e.g. SERM, etc.) on a hormone receptor-positive cancer (e.g. an ERα-positive cancer) can be evaluated, and can be supplementally evaluated.

### BRIEF DESCRIPTION OF DRAWINGS

FIG. 1 is a view schematically showing the function of Fbxo22 in cofactor dynamics to ERα. First panel: ERα is present as a monomer in the absence of E2 and is dissociated from SRC and KDM4B. Second panel: an E2-bound ERα dimer forms a complex with KDM4B and SRC in Fbxo22-positive breast cancer cells in the presence of E2. As a result of the formation of this complex, ERα signaling is activated. Third panel: SCF^{Fbxo22} specifically ubiquitinates KDM4B that forms a complex with SERM-bound ERα in the presence of SERM, and induces decomposition. When KDM4B is decomposed in Fbxo22-positive breast cancer cells, dissociation of SRC from ERα and interaction between ERα and N—CoR are promoted. As a result of the dissociation of the SRC complex, ERα signaling is inactivated. Fourth panel: In Fbxo22-negative cells, SERM-bound ERα still interacts with SRC and KDM4B in the presence of SERM, and this complex activates ERα signaling.

FIG. 2 shows the results obtained by examining the influence of the antagonistic activity of TAM on proteasome-dependent proteolysis. FIG. 2A shows an experimental schedule (upper view). MCF-7 cells that had been in an E2-depleted condition for 72 hours were cultured in a medium supplemented with E2 (10 nM) in the presence or absence of MG132 (10 μg/ml) for 6 hours, and then, were cultured in a medium supplemented with 4-OHT (100 nM). Using total RNA derived from individual cells recovered over time, qPCR was carried out. The data are shown with the mean value±standard deviation of the data of the three independent experiments. **** p<0.001, and *** p<0.005. FIG. 2B shows that immunoprecipitation and immunoblotting were carried out with the antibodies shown in the figure, using a cell nucleus extract after the culture for 12 hours following addition of E2. FIG. 2C shows that MCF-7 cells that had been in an E2-depleted condition for 72 hours were cultured in a medium supplemented with E2 (10 nM) for 18 hours (E2), or were cultured in the presence or absence of MG132 (10 μg/ml) for 6 hours, and were then cultured in an E2-depleted condition (E2-dep) for 12 hours. Using total RNA prepared from the treated cells, qPCR was carried out. The data are shown with the mean value±standard deviation of the data of the three independent experiments. ***p<0.005. FIG. 2D shows that immunoprecipitation and immunoblotting were carried out with the antibodies shown in the figure, using the cell nucleus extract treated in the same manner as that in FIG. 2C. FIG. 2E shows that MCF7 cells expressing Dox (doxycycline)-induced shControl or shKDM4B were left in an E2-depleted condition in the presence of doxycycline (1 μg/ml) for 72 hours, and thereafter, were treated with E6 (10 nM) for 6 hours. Using the nucleus extract, immunoprecipitation and immunoblotting were carried out.

FIG. 3 shows the results obtained by analyzing the formation of a complex between Fbxo22, and ERα and KDM4B. FIG. 3A shows that HeLa cells expressing Dox-induced shControl or shKDM4B were treated with doxycycline (1 μg/ml), and that the cell lysate thereof was then subjected to immunoblotting over time. FIG. 3B shows that MCF-7 cells expressing Dox-induced shControl or shKDM4B that had been cultured in the presence of doxycycline (1 μg/ml) for 24 hours were treated with 50 μg of cycloheximide (CHX). Immunoblotting was carried out on the cell lysate over time, and the band strength of KDM4B was then determined by Image J. The data are shown with the mean value±standard deviation of the data of the three independent experiments. ***p<0.001. FIG. 3C shows that MCF-7 cells expressing Dox-induced FLAG-HA-Fbxo22 were cultured in the presence or absence of doxycycline (1 μg/ml) for 48 hours, and were then treated with MG132 (10 μg/ml) for 4 hours. Using anti-FLAG M2 affinity gel and anti-HA affinity gel, immunoprecipitation was carried out on the cell lysate. Then, immunoblotting was carried out on the obtained immunoprecipitate. FIG. 3D shows that MCF-7 cells were treated with MG132 (10 μg/ml) for 4 hours, and that immunoprecipitation and immunoblotting were then carried out on the cell lysate. FIGS. 3E and 3F show that MCF-7 cells expressing various types of Dox-induced shRNAs were cultured in the presence of doxycycline (1 μg/ml) for 48 hours. Thereafter, immunoprecipitation was carried out on the cell lysate. FIG. 3G shows that MCF-7 cells expressing wild-type FLAG-Fbxo22 (Wt), FIST-N-deficient mutant (AFN) or FIST-C-deficient mutant (AFC) were treated in the same manner as that in FIG. 3C. Using anti-FLAG M2 affinity gel, immunoprecipitation was carried out on the cell lysate. Then, immunoblotting was carried out on the obtained immunoprecipitate. FIG. 3H shows that MCF-7 cells expressing Dox-induced FLAG-Fbxo22 were treated in the same manner as that in FIG. 3C. Using anti-FLAG M2 affinity gel and an anti-ERα antibody, immunoprecipitation was continuously carried out on the cell lysate. Then, immunoblotting was carried out on the obtained immunoprecipitate. FIG. 3I shows that MCF-7 cells expressing Dox-induced FLAG-Fbxo22 were cultured in a medium excluding E2 in the presence or absence of doxycycline (1 μg/ml) for 72 hours to obtain an E2-depleted condition. Thereafter, the resulting cells were treated in the presence of MG132 (10 μg/ml) and in the presence or absence of 0.1 nM, 1 nM or 10 nM E2 for 6 hours. Thereafter, immunoprecipitation and immunoblotting were carried out on the cell lysate. FIG. 3J shows that MCF-7 cells expressing Dox-induced FLAG-Fbxo22 were cultured in a medium excluding E2 in the presence or absence of doxycycline (1 μg/ml) for 72 hours. Thereafter, the resulting cells were treated in the presence of MG132 (10 μg/ml) and in the presence of E2 (10 nM) and/or 1 nM, 10 nM, or 100 nM 4-OHT for 6 hours. Thereafter, immunoprecipitation and immunoblotting were carried out on the cell lysate.

FIG. 4 shows the results obtained by analyzing the ubiquitination of KDM4B by SCF^{Fbxo22}. FIG. 4A shows that immunoblotting was carried out on an MCF-7 cell lysate expressing Fbxo22 and/or ERα, using various types of antibodies. FIG. 4B shows that Fbxo22 knockout HeLa cells were transfected with various types of plasmids, and were then treated with MG132, and thereafter, the resulting cells were lyzed under degeneration conditions and were then subjected to a Strep-Tactin pull down assay. Thereafter, immunoblotting was carried out on the precipitate. FIG. 4C shows that the Fbxo22 knockout HeLa cells expressing various types of genes shown in FIG. 4B were treated in the presence or absence of E2 and 4-OHT, and were then subjected to immunoblotting. St2-KDM4B: tandem strep-II-tagged KDM4B

FIG. 5 shows the results obtained by clarifying that Fbxo22 via AF1 activity is necessary for the antagonistic activity of 4-OHT. FIG. 5A shows an experimental schedule (upper view). MCF-7 cells expressing Dox-induced shFbxo22 were cultured in the presence of doxycycline (1 μg/ml) for 72 hours to obtain an E2-depleted condition, and were then treated with E2 (10 nM) for 6 hours, and thereafter, the resulting cells were treated with 4-OHT (100 nM). Using total RNA prepared from the cells over time, qPCR was carried out. The data are shown with the mean value±standard deviation of the data of the three independent experiments. *p<0.05, and ****p<0.001.

FIG. 5B shows that immunoprecipitation and immunoblotting were carried out on a cell nucleus extract after the culture for 12 hours, which was treated in the same manner as that in FIG. 5A. FIG. 5C shows that U2OS cells expressing wild-type ERα or Δ44 mutant ERα were treated in the same manner as that in FIG. 5A. ****p<0.001. FIG. 5D shows that U2OS-LacO-I-SceI-TetO cells expressing Dox-induced shControl or shFbxo22 and FLAG-KDM4B were transfected with a YFP-SRC-1 plasmid and a CFP-ERα lpha-Lac plasmid, and were then treated with doxycycline (1 μg/ml) in a medium excluding E2 for 72 hours. The thus treated cells were treated in the presence or absence of E2 (10 nM) and/or 4-OHT (100 nM) for 2 hours, and were then fixed with 4% formaldehyde. The obtained cells were immunostained with anti-FLAG antibodies. Representative images are shown. FIG. 5E shows that foci, in which CFP-ERα lpha-Lac, YFP-SRC-1 and FLAG-KDM4B co-localized with one another, were counted. The data are shown with the mean value±standard deviation of the data of the three independent experiments. **p<0.01.

FIG. 6 shows studies regarding the influence of Fbxo22 on Toremifene and Fulvestrant. FIG. 6A shows that MCF-7 cells expressing various types of Dox-induced shRNAs were cultured in the presence of doxycycline for 72 hours to obtain an E2-depleted condition, were then treated with E2 for 6 hours, and were then treated with 4-OHT, Toremifene (Tor) or Fulvestrant (Ful) for 12 hours. Total RNA was prepared from the cells treated with various drugs, and qPCR was carried out. The data are shown with the mean value±standard deviation of the data of the three independent experiments. **p<0.01, and ****p<0.001. FIG. 6B shows that immunoprecipitation and immunoblotting were carried out on cell lysates treated in the same manner as that in FIG. 6A, using various types of antibodies.

FIG. 7 shows the results obtained by the genome-wide analysis of the influence of Fbxo22 on dissociation of SRC-3 from an ERα/SRC3-binding site in the presence of TAM. FIG. 7A shows that ERα-binding sites having a sequence center within 0.1 kb from one another were identified as common peaks in 4 data sets (wild-type MCF-7 cells stimulated by E2 or E2+4-OHT, and Fbxo22-depleted MCF-7 cells stimulated by E2+4-OHT). In addition, SRC-3-binding sites were identified as common peaks in 2 data sets (wild-type MCF-7 cells stimulated by E2 and Fbxo22-depleted MCF-7 cells stimulated by E2). FIG. 7B shows an SRC-3-binding tag count in the 410 SRC-3 common peaks shown in FIG. 7A. ****p<0.001, and n.s.: No significant difference. FIG. 7C shows the heat maps of ERα and SRC-3 in the MCF-7 cells. FIG. 7D shows the gene browser snapshots of the ChIP-seq of ERα and SRC-3 (p160) in the GREB1 and IGFBP4 gene loci of the MCF-7 cells.

FIG. 8 shows studies regarding the influence of Fbxo22 on the ability of 4-OHT to inhibit proliferation of breast cancer cells. FIG. 8A shows that doxycycline (1 μg/ml) was added to MCF-7 cells expressing Dox-induced shControl or shFbxo22 and the obtained mixture was then cultured in the presence or absence of Dox-induced FLAG-Fbxo22 for 72 hours to obtain an E2-depleted condition. Thereafter, the cells were treated with E2 (10 nM) in the presence or absence of 4-OHT (100 nM) for 6 hours, and a quantitative colony formation assay was carried out. Representative colony images (upper view) and the results of the assay are shown. The data are shown with the mean value±standard deviation of the data of the three independent experiments. FIG. 8B shows that MCF-7 cells expressing various types of Dox-induced shRNAs were treated in the same manner as that in FIG. 8A, and a quantitative colony formation assay was carried out. The data are shown with the mean value±standard deviation of the data of the three independent experiments. FIG. 8C shows that the tumor volume of control T47D cells (n=5) or Fbxo22-KO T47D cells (n=5), which had been transplanted into the mammary gland fat pad of NOD/Scid mice, was measured 2 weeks after the embedding of an E2 pellet or 4 weeks after the embedding of a tamoxifen pellet. *p<0.05. FIG. 8D shows that the mice were euthanized 6 weeks after the cell transplantation, the tumor was then excised, and the weight thereof was then measured. *p<0.05. FIG. 8E shows the excised tumors. FIG. 8F shows that an anti-Ki-67 antibody and cleaved caspase 3-specific antibody were used for paraffin-embedded tumor sections derived from five mice into which wild-type T47D cells (Wt) or Fbxo22-KO (KO) T47D cells had been transplanted, so as to carry out an immunohistochemical analysis. Ki-67-positive and cleaved caspase 3-positive cells were counted, and the counted numbers were then normalized with respect to the cell nucleus in each section. *p<0.05, ***p<0.05.

FIG. 9 shows the results showing that the expression level of Fbxo22 becomes a prognostic factor for ERα-positive breast cancer. FIGS. 9A and 9B show representative immunohistochemical staining images of a normal mammary gland (FIG. 9A), an Fbxo22-positive mammary gland of human breast cancer (FIG. 9B, left view), and an Fbxo22-negative mammary gland (FIG. 9B, right view). Scale bar: 20 μm. FIGS. 9C to 9F show Kaplan-Meier survival curves formed by stratifying ERα-positive/HER2-negative breast cancer (FIG. 9C), Luminal A (low Ki-67) breast cancer (FIG. 9D), lymph node metastasis-negative breast cancer (FIG. 9E), and tamoxifen-administered breast cancer (FIG. 9F) by the expression of Fbxo22. The P value and the hazard ratio were calculated according to a log-rank test.

FIG. 10 shows stratification by Fbxo22 and other clinicopathological factors. FIG. 10A shows distribution of the percentage of Fbxo22-positive cells in 163 ERα-positive/HER2-negative breast cancer cases. FIGS. 10B and 10C shows the results obtained by stratifying grade 1 ERα-positive/HER2-negative breast cancer (FIG. 10B) and tamoxifen-administered breast cancer (FIG. 10C) by the expression of Fbxo22. FIGS. 10D to 10F show Kaplan-Meier survival curves formed by stratifying all T2 grade ERα-positive/HER2-negative breast cancers, based on Ki-67 status (≤10% vs. 20%≤) (FIG. 10D), lymph node metastasis (positive vs. negative) (FIG. 10E), or grade (1 vs. 2/3) (FIG. 10F).

FIG. 11 shows studies regarding the expression level of Fbxo22 in the normal endometrium. FIG. 11A shows the results obtained by confirming the expression levels of Fbxo22 and Ki-67 in the proliferative phase and the secretory phase. FIGS. 11B to 11D are graphs showing the expression level of Fbxo22 (FIG. 11B) with H-Score, and expression levels of Ki-67 (FIG. 11C) and PgR (FIG. 11D) with positive cell percentage, in the endometrium proliferative phase (P, 8 cases), the secretory phase (S, 6 cases), and the early secretory phase (ES, 8 cases).

FIG. 12 shows studies regarding the expression level of Fbxo22 in endometrial cancer. FIG. 12A shows the results obtained by confirming the expression levels of Fbxo22 and Ki-67 in premalignant endometrial hyperplasia (EH), atypical endometrial hyperplasia (AEH) as a non-invasive early cancer, and endometrial cancer (EC), according to immunostaining with antibodies. FIG. 12B are graphs showing the expression level of Fbxo22 in EH (30 cases), AEH (29 cases) and EC (30 cases) with H-Score.

FIG. 13 shows the results of the immunostaining of Fbxo22, Ki-67 and ER (Fbxo22, Ki-67 and ER, respectively) and the results of the HE staining (HE), in the endometrial tissues of AEH cases, in which EH was also present. In an identical case, Fbxo22 was positive in the gland duct with EH, and Fbxo22 was negative in the gland duct with AEH.

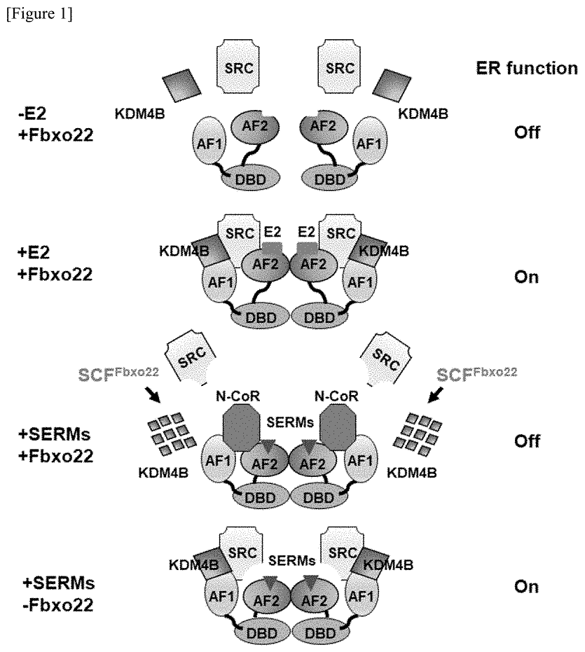

### DESCRIPTION OF EMBODIMENTS

The present invention relates to: a method for determining the prognosis of a cancer (or for predicting the prognosis of a cancer), or a method for assisting the determination of the prognosis, in which the expression level of Fbxo22 in hormone receptor-positive cancer cell tissues is used as an indicator; and a method for evaluating the sensitivity of the cancer to antihormone therapy or the therapeutic effects of an anti-hormonal agent on the cancer, based on the results obtained by determining the prognosis (namely, the results obtained by measuring the expression level of Fbxo22 in the cancer tissues), or a supplemental evaluation method.

Specifically, a first embodiment of the present invention relates to a method for determining the prognosis of a hormone receptor-positive cancer, or a method for assisting the determination of the prognosis, which comprises the following steps (a) and (b):

(a) a step of detecting Fbxo22-positive cancer cells in a sample derived from the cancer tissues; and

(b) a step of calculating the percentage of the Fbxo22-positive cancer cells to cancer cells present in the sample.

In the above-described determination method, when the percentage of the Fbxo22-positive cancer cells calculated in the step (b) is less than a predetermined percentage, the prognosis of the hormone receptor-positive cancer is determined to be poor. Besides, since the above-described steps (a) and (b) can also be carried out by professionals other than doctors, these steps are also constituent elements of a method for assisting the determination of the prognosis, which is for use in providing information necessary for the determination of prognosis by doctors.

Herein, the term “prognosis” has the same meanings as those commonly used in the medical field, and thus, is not particularly limited. For example, the prognosis means a point of view regarding a predictable medical condition (health condition) or a future state of disease and/or wound. In addition, the term “poor prognosis” means, for example, a reduction in the survival rate, an increased risk of recurrence, and/or the possible metastasis of a tumor to another site.

The “hormone receptor-positive cancer” of the first embodiment of the present invention is a cancer expressing a hormone receptor, and examples of the hormone receptor-positive cancer may include: ERα (estrogen receptor α)-positive cancers such as ERα-positive breast cancer and ERα-positive endometrial cancer; and AR (androgen receptor)-positive cancers such as AR-positive prostate cancer. It has been reported that KDM4B plays an important role also in androgen signaling (Coffey et al., Nucleic Acids Res. 41: 4433-4446, 2013). Accordingly, it is considered that the method for determining prognosis (or the method for assisting the determination) of the present invention can also be utilized in an antihormone therapy using SARM (selective androgen receptor modulator) for an AR-positive prostate cancer, as well as an ERα-positive breast cancer.

As mentioned above, Fbxo22 is an F-box protein consisting of three functional domains (F-box, FIST-N, and FIST-C), which forms a complex with SCF and functions as ubiquitin ligase. In the present description, when the term “Fbxo22” is used, it means an Fbxo22 protein. Information such as the amino acid sequence of Fbxo22 (GenBank no.: AAH20204.2, SEQ ID NO: 6) and a nucleic acid sequence encoding this protein (GenBank no.: BCO20204.1, SEQ ID NO: 7) has been disclosed in already published database and the like, a person skilled in the art could readily obtain the information.

The first embodiment of the present invention includes a step of detecting Fbxo22 in cancer tissues (cancer cells). In this case, a means for collecting a sample comprising cancer tissues from a target patient whose prognosis is to be determined can be carried out any methods easily selected by a person skilled in the art, such as, for example, a needle biopsy of obtaining a cell sample using a puncture needle, or an incision biopsy of obtaining an affected tissue section by surgical incision.

Fbxo22-expressing cells can be detected in a sample comprising cancer tissues (cancer cells) by a method that can be easily selected by a person skilled in the art. For example, when Fbxo22 expressed in cancer cells comprised in a sample is detected and the expression level thereof is then examined according to an immunohistochemical method, a suitable tissue specimen or cytologic specimen may be produced, and studies may be then conducted. As a method of producing a tissue specimen or a cell specimen, any known methods may be applied. For example, collected tissues and the like may be fixed with formalin, etc., may be then embedded in paraffin to produce a section, which may be subjected to immunohistochemical staining, so that the expression level of Fbxo22 may be examined. Alternatively, one of cytologic methods, Liquid Based Cytology (LBC) may be applied to examine the expression level of Fbxo22. According to the LBC method, a collected cytologic specimen (tumor cell sample) is stirred and/or dispersed in a dispersed solution (preservative solution), and then, the cells are recovered and are thinly transcribed and smeared on a slide glass, so that the cells are immobilized, and thereafter, antibody staining or the like is carried out to examine the amount of the stained Fbxo22.

In order to detect Fbxo22 expressed in the collected tissues or cells according to an immunohistochemical method, an antibody reacting against Fbxo22 (anti-Fbxo22 antibody) can be used. As such an anti-Fbxo22 antibody, either an antibody produced by a person who carries out the present invention, or a commercially available antibody (for example, Gene Tex[N3C3], Sigma[FF-7], etc.) can be used. Moreover, the anti-Fbxo22 antibody may be either a monoclonal antibody or a polyclonal antibody. Furthermore, the anti-Fbxo22 antibody does not need to be an antibody having a complete body, but it may be an antibody fragment comprising a CDR region, etc., or a genetically engineered antibody. Such an antibody fragment is not particularly limited, as long as it can bind to Fbxo22 expressed in cells and can be used in immunohistochemical staining. Examples of the antibody fragment may include Fab, Fab′, F(ab′)2, Fv (a variable fragment of antibody), a single chain antibody (a heavy chain, a light chain, a heavy chain variable region, a light chain variable region, etc.), scFv, diabody (an scFv dimer), dsFv (a disulfide-stabilized variable region), and a peptide comprising CDR at least as a portion thereof, all of which are peptide fragments comprising a partial region of the anti-Fbxo22 antibody.

When a tissue or cell sample, etc. collected from a patient is immunostained with an anti-Fbxo22 antibody, such immunostaining can be carried out: by allowing a suitable label to bind to the anti-Fbxo22 antibody, or to a secondary antibody that is to be bound to the anti-Fbxo22 antibody, if the anti-Fbxo22 antibody is used as a primary antibody; and then by visualizing the label. For example, when peroxidase is used in labeling, diaminobenzidine (DAB), aminomethylcarbazole (ACE) or the like is used as a chromogenic substrate, whereas when alkaline phosphatase is used in labeling, 5-bromo-4-chloro-3-indoxyl phosphate/nitroblue tetrazolium chloride (BCIP/NBT) or the like is used as a chromogenic substrate, so that the staining can be carried out.

Next, when the presence or absence of Fbxo22 expression in cells is evaluated according to an immunohistochemical method, the evaluation method is not particularly limited. For example, the evaluation can be carried out as follows. Specifically, a tissue or cell specimen that has been stained at a low magnification is observed under a microscope, a region having the highest staining intensity is selected. Subsequently, the selected region is observed under a high-power field, and 100 cells are then selected as observation targets. The staining intensity of the nucleus of each of the thus selected 100 cells is classified, for example, into the following 4 classifications.

No staining: Cells whose nuclei are not stained or whose nuclei are slightly stained at the same level as the background.
Low degree: Cells that are not distinguished from non-stained cells when they are observed at a low magnification, but when they are observed at a high magnification, the staining of the nuclei thereof can be slightly confirmed.
Moderate degree: Cells, in which the staining of the nuclei thereof can be confirmed at a low magnification, and the staining of the nuclei thereof can be completely confirmed at a high magnification.
High degree: Cells, in which the staining of the nuclei thereof can be completely confirmed at a low magnification.

The selected 100 cells, in which the staining intensity of the nuclei thereof is at a moderate level or high, are determined to be “Fbxo22-positive cells,” and the percentage thereof is calculated. Although the percentage is somewhat fluctuated depending on the detection method, when the percentage is less than 30%, less than 20%, less than 10%, preferably less than 5%, or more preferably less than 1%, the stainability by the anti-Fbxo22 antibody is determined to be “negative.” In addition, it can be determined that the prognosis of a cancer, from which the “negative” sample is derived, is highly likely to be poor. Besides, the percentage of the “Fbxo22-positive cells” in the case of determining to be “negative” is desirably set, such that the negative cases (cases determined to be “negative”) can be approximately 30% of all cases.

Moreover, the stainable state of the collected sample may be photographed by a camera or the like, so that an image of the stained cells may be obtained. Thereafter, the image may be subjected to electronic data processing, and may be analyzed. Otherwise, the staining intensity obtained from the image may be quantified, and may be converted to a numerical form, so that the above-described 4 stages of staining levels may be evaluated. For example, a high magnification field image (from 200- to 400-fold) of a stained main cancer cell population is photographed through a microscope, and the colors of Fbxo22 staining in the photographed image are divided classified into RGB colors, namely, red, green and blue according to a bioimaging analysis system, etc. The stainabilities of the colors are each indicated with hue and binarized. The stained positive area on the photographed image is obtained by subtraction of a negative area that is non-specifically stained with a negative antibody (control antibody), and the percentage of the positive area may be defined to be the percentage of the “Fbxo22-positive cells.”

Alternatively, according to, what is called, a hybridization method, the mRNA of Fbxo22 expressed in cancer cells in a sample may be detected, and the expression status of Fbxo22 may be monitored. The available hybridization method may be, for example, an in situ hybridization method. A labeled probe complementary to the mRNA of Fbxo22, such as, for example, a radiolabeled probe, a digoxigenin (DIG) probe, a fluorescent labeled (FITC, RITC, etc.) probe, or the like is used to a tissue section or a cell specimen obtained from cancer tissues serving as targets of prognosis determination, so that the mRNA level of Fbxo22 in the sample section or specimen can be detected. For evaluation of the mRNA level of Fbxo22 in the sample, signals obtained from the labeled probe are observed under a microscope, the observed cells are then classified into 4 stages, as described above, based on signal strength from the label. Thereafter, the percentage of the cells, in which the signal strength is at a moderate level or higher, is calculated, and it can be used as an indicator for determining prognosis or assisting the determination of prognosis.

What is more, in addition to the immunohistochemical method, as a method for detecting the expression level of Fbxo22 in tumor cells in a sample, a quantitative RT-PCR (real time PCR) method may be applied to detect the expression level of Fbxo22 mRNA in the sample.

Information regarding the thus obtained percentage of Fbxo22-positive cells in a test target sample can be used as a material for determining the prognosis of a cancer, from which the test target sample is derived.

The results obtained by the method for determining prognosis or the method for assisting the determination according to the first embodiment can be used as a base for the determination of the sensitivity of the cancer to an antihormone therapy. That is to say, the case where a cancer is determined to have a poor prognosis or to be highly likely to have a poor prognosis by the method for determining prognosis or the method for assisting the determination of the present invention is a case where Fbxo22 is not expressed or is expressed in an extremely small amount in the cancer cells. In such a case, if an antihormone therapy (for example, the treatment of an ERα-positive breast cancer with SERM (selective estrogen receptor modulator) or the treatment of an AR-positive prostate cancer with SARM (selective androgen receptor modulator)) is performed on the cancer, it can be evaluated that no therapeutic effects are obtained, or that a recurrence risk is rather increased (see Examples).

In view of the foregoing, a second embodiment of the present invention relates to a method for evaluating the therapeutic effects of an anti-hormonal agent on a hormone receptor-positive cancer based on the results obtained by the method for determining prognosis or the method for assisting the determination of prognosis according to the first embodiment of the present invention, or a method for supplementally evaluating the therapeutic effects.

Examples of the anti-hormonal agent used in the embodiment of the present invention may include: SERM, when the hormone receptor-positive cancer is an ERα-positive cancer such as an ERα-positive breast cancer or an ERα-positive endometrial cancer; and SARM, when the hormone receptor-positive cancer is an AR-positive cancer.

Herein, SERM and SARM mean a selective estrogen receptor modulator and a selective androgen receptor modulator, respectively. SERM and SARM are generic names of compounds exhibiting an agonistic action or an antagonistic action in organs or tissues.

Examples of the SERM used in the second embodiment may include tamoxifen, toremifene, lasofoxifene, arzoxifene, ospemifene, raloxifene, and derivatives thereof. Examples of the SARM may include andarine, ostarine, and derivatives thereof.

A third embodiment of the present invention relates to a kit for determining the prognosis of hormone receptor-positive cancers such as an ERα-positive breast cancer, an ERα endometrial cancer and an AR-positive prostate cancer, or for assisting the determination of the prognosis; or for evaluating the therapeutic effects of an anti-hormonal agent such as SERM or SARM against these cancers, or for supplementally evaluating the therapeutic effects. As mentioned above, the first and second embodiments of the present invention provide, respectively, a method for determining the prognosis of a hormone receptor-positive cancer, or for assisting the determination of the prognosis; and a method for evaluating the therapeutic effects of an anti-hormonal agent such as SERM or SARM on the hormone receptor-positive cancer, wherein the therapeutic effects are obtained when the cancer is treated with the anti-hormonal agent, or for supplementally evaluating the therapeutic effects, in each of which the expression level of Fbxo22 in the hormone receptor-positive cancer is used as an indicator. Accordingly, elements for measuring the expression level of Fbxo22 in cancer cells in a sample, such as, for example, an anti-Fbxo22 antibody, and a probe, a primer or the like used to measure the expression level of Fbxo22 mRNA, have the intended use for determining the prognosis of a hormone receptor-positive cancer and for evaluating the therapeutic effects of an anti-hormonal agent on the cancer. This intended use is disclosed for the first time in the present application. The kit of the third embodiment of the present invention comprises, as essential constituent elements, an anti-Fbxo22 antibody, and a probe, a primer, and the like used to measure the expression level of Fbxo22 mRNA. In addition to these essential constituent elements, the kit of the third embodiment of the present invention may further comprise auxiliary constituent elements such as, for example, a fixing agent necessary for immunostaining tissues or cells serving as diagnostic targets, such as formalin, and a chromogenic substrate, a buffer and the like necessary for performing immunohistochemical staining or quantitative RT-PCR.

When an English translation of the present description includes singular terms with the articles “a,” “an,” and “the,” these terms include not only single items but also multiple items, unless otherwise clearly specified from the context.

Hereinafter, the present invention will be further described in the following examples. However, these examples are only illustrative examples of the embodiments of the present invention, and thus, are not intended to limit the scope of the present invention.

### EXAMPLES

### 1. Methods

1-1. Antibodies and shRNAs

Information regarding the shRNAs and antibodies used in the present Examples are shown in the following Table 1 and Table 2, respectively.

TABLE 1

| Target gene | Sequence |
|---|---|
| Fbxo22 | GGAATTGTAGTGACTCCAATG (SEQ ID NO: 2) |
| KDM4B | GGAAGGACATGGTCAAGAT (SEQ ID NO: 3) |
| Luciferase | CGTACGCGGAATACTTCGA (SEQ ID NO: 4) |

TABLE 2

| | Animal | |
|---|---|---|
| Antibody | species | Source |
| Anti-alpha-beta-Actin | Mouse | Neomarkers, Fremont, CA |
| (DMIA + BMIB) | | |
| Anti-beta-Actin(6276) | Mouse | Abcam, Cambridge, United Kingdom |
| Anti-EGFP (0153-3) | Rat | MBL, Nagoya, Japan |
| Anti-ERalpha (HC-20) | Rabbit | Santa Cruz Biotechnologies, |
| | | Santa Cruz, CA |
| Anti-ERalpha (H-184) | Rabbit | Santa Cruz Biotechnologies, |
| | | Santa Cruz, CA |
| Anti-Fbxo22 (N3C3) | Rabbit | GeneTex, Irvine, CA |
| Anti-Fbxo22 (FF-7) | Mouse | Santa Cruz Biotechnologies, |
| | | Santa Cruz, CA |
| Anti-FLAG (M2) | Mouse | Sigma, St. Louis, MD |
| Anti-HA (3F10) | Rat | Roche, Basel, Switzerland |
| Anti-HA (12CA5) | Mouse | Boehringer, Mannheim, Germany |
| Anti-KDM4A | Mouse | UC Davis/NIH NeuroMab |
| (N154/32) | | Facility, Davis, CA |
| Anti-KDM4B (D7E6) | Rabbit | Cell Signaling Technology, |
| | | Boston, MA |
| Anti-KDM4C (27532) | Rabbit | Abcam, Cambridge, United Kingdom |
| Anti-KDM4D (63199) | Rabbit | Abcam, Cambridge, United Kingdom |
| Anti-LC3 (2775) | Rabbit | Cell Signaling Technology, Boston, MA |
| Anti-Myc (N262) | Rabbit | Santa Cruz Biotechnologies, |
| | | Santa Cruz, CA |
| Anti-Myc (17355) | Mouse | Abcam, Cambridge, United Kingdom |
| Anti-NcoR (5948) | Rabbit | Cell Signaling Technology, Boston, MA |
| Anti-SRC3 (5E11) | Rabbit | Cell Signaling Technology, Boston, MA |
| Anti-StrepII | Rabbit | Abnova, Walnut, CA |
| (PAB16603) | | |
| Anti-TFEB (4240) | Rabbit | Cell Signaling Technology, Boston, MA |

### 1-2. Cell Culture

Cell culture and treatments with various drugs were carried out in accordance with the method described in Johmura et al., Molecular Cell 55: 73-84, 2014.

MCF-7 cells, T47D cells, ZR75-1 cells, U2OS cells, U2OS-LacO-I-SceI-TetO (Burgess et al., Cell reports. 9: 1703-17, 2014), or 293T cells were cultured in DMEM (Invitrogen) supplemented with 10% fetal bovine serum (FBS).

Estradiol (E2) (Sigma-Aldrich), cycloheximide (Sigma-Aldrich), 4-hydroxytamoxifen (4-OHT) (Sigma-Aldrich), Fulvestrant (Sigma-Aldrich), Toremifene (Sigma-Aldrich), and MG132 (Sigma-Aldrich) were used in concentrations of 10 nM, 50 μg/ml, 100 nM, 10 μg/ml, 100 nM, and 100 nM, respectively.

To create an E2-depleted condition, the following operations were carried out. The cells were washed with PBS three times, and were then cultured in phenol red-non-added DMEM (supplemented with 5% charcoal-stripped FBS) for 72 hours.

### 1-3. Colony Formation Assay

The cells (5×10²) were seeded on a 6-well plate, and were then cultured for 2 weeks. Thereafter, colonies were immobilized with methanol/acetic acid (1:1) for 15 minutes, and were then stained in 20% methanol/PBS containing 0.4% trypan blue (Sigma) for 15 minutes, followed by counting.

### 1-4. Construction of Plasmid

A lentivirus-based shRNA construct and a Tet-on-induced lentivirus construct were produced according to the method described in Johmura et al., Molecular Cell 55: 73-84, 2014.

In order to prepare a lentivirus-based shRNA construct, 19-21 bp shRNA-coding fragments having a loop sequence (SEQ ID NO: 1: 5′-ACGTGTGCTGTCCGT-3′) (Table 1: Fbxo22 (SEQ ID NO: 2), KDM4B (SEQ ID NO: 3), and Luciferase (SEQ ID NO: 4)) were inserted into pENTR4-H1 cleaved with AgeI/EcoRI. Subsequently, in order to insert HltetOxl-shRNA into a lentiviral vector, the obtained pENTR4-H1-shRNA vector was mixed with a CS-RfA-ETBsd vector or a CS-RfA-ETPuro vector, and the mixture was then reacted using Gateway LR clonase (Invitrogen).

In order to construct a Tet-on induced lentivirus construct, a pENTR-1A vector (Invitrogen) comprising the sequence of Flag, HA, FLAG-HA or EGFP was cleaved with BamHI/NotI. Thereafter, a BamHI/NotI fragment comprising the cDNA of wild-type human/mouse Fbxo22 or each variant thereof, or the cDNA of wild-type human KDM4B was amplified by PCR, and the resultant was then inserted into the above-described BamHI/NotI cleaved site. The obtained plasmid was mixed with a CS-IV-TRE-RfA-UbC-Puro vector or a CS-IV-TRE-RfA-UbC-Hygro vector, and the mixture was then reacted using Gateway LR clonase (Invitrogen), so as to prepare a lentivirus plasmid.

pcDNA3-(HA-Ub)x6 comprising, in tandem, six HA-tagged ubiquitin-coding sequences was produced by the method described in Nishikawa et al., The Journal of biological chemistry 279: 3916-3924, 2004. pcDNA3-St2-KDM4B was produced using KDM4B cDNA sub-cloned into pcDNA3 retaining the following oligonucleotide corresponding to a Strep II epitope:

(SEQ ID NO: 5)
TGGAGCCATCCTCAGTTCGAGAAAGGTGGCGGTTCTGGCGGAGGGTCGGGC
GGCTCCGCCTGGAGTCACCCTCAGTTTGAGAAA-3′.

### 1-5. Preparation of Virus and Infection

Preparation of lentivirus and infection of the cells with the prepared virus were carried out according to the method described in Johmura et al., Molecular Cell 55: 73-84, 2014. Lentiviruses expressing shRNA or various types of genes were prepared by being co-transfected into 293T cells according to a calcium phosphate co-precipitation method, using pCMV-VSV-G-RSV-RevB, pCAG-HIVgp, and CS-RfA-ETBsd, CS-RfA-ETPuro, CS-IV-TRE-RfA-UbC-Puro, CS-IV-TRE-RfA-UbC-Hygro or CSII-CMV-MCS. The cells infected with the lentivirus were treated with 10 μg/ml blasticidin (Invitrogen) and/or 2 μg/ml puromycin (Sigma-Aldrich) for 2 or 3 days. doxycycline (Sigma-Aldrich) was added to the resulting cells to a concentration of 1 μg/ml, so as to induce the expression of each shRNA or each gene.

### 1-6. Immunoprecipitation and Immunoblotting

Immunoprecipitation and immunoblotting were carried out according to the method described in Johmura et al., Molecular Cell 55: 73-84, 2014. The cells were dissolved in a TBSN buffer (20 mM Tris-Cl (pH 8.0), 150 mM NaCl, 0.5% NP-40, 5 mM EGTA, 1.5 mM EDTA, and 0.5 mM Na₃VO₄). The obtained cell lysate was centrifuged at 4° C. at 15,000×g for 20 minutes, and was then used in immunoprecipitation using an antibody.

Regarding immunoprecipitation of ERα and KDM4B, a nuclear extract was prepared according to the method described in Hirokawa et al., Cancer Res. 74: 3880-3889, 2014. Cell pellets were suspended in buffer A (10 mM Hepes-KOH pH 7.9, 10 mM KCl, 1.5 mM MgCl₂, 0.5 mM DTT, 0.5 mM PMSF, and protease inhibitor cocktail (Nakalai Tesque)) having five times the volume of the cell pellets, and the suspension was then incubated on ice for 5 minutes. Thereafter, the cells were centrifuged at 4° C. at 500×g for 5 minutes, and the obtained cell pellets were then suspended in buffer A having two times the volume of the cell pellets for homogenization. The homogenized cell suspension was centrifuged at 4° C. at 4,000×g for 5 minutes, and a cell nucleus was recovered in a precipitate. To the recovered nucleus, an equal amount of buffer B (20 mM Hepes-KOH pH 7.9, 600 mM KCl, 1.5 mM MgCl₂, 0.2 mM EDTA, 25% glycerol, 0.5 mM DTT, 0.5 mM PMSF, and protease inhibitor cocktail) was added and suspended, and the obtained mixture was then blended at 4° C. for 30 minutes, using a rotator. The nuclear extract was centrifuged at 4° C. at 16,000×g for 15 minutes, and was then recovered in a supernatant. The nuclear extract was dialyzed against buffer C (20 mM Hepes-KOH pH 7.9, 100 mM KCl, 0.2 mM EDTA, 20% glycerol, 0.5 mM DTT, and 0.5 mM PMSF). After completion of the dialysis, the nuclear extract was centrifuged at 4° C. at 16,000×g for 30 minutes, and the remaining precipitate was then eliminated.

Regarding the total cell lysate, the cells were directly dissolved in Laemmli-buffer (2% SDS, 10% glycerol, 5% 2-mercaptoethanol, 0.002% bromophenol blue, and 62.5 mM Tris HCl pH 6.8).

A protein (20 to 50 μg) was separated from the total cell lysate by SDS-PAGE, and was then transferred into a PVDF membrane (Millipore), which was then used in immunoblotting using various types of antibodies.

### 1-7. Quantitative RT-PCR

Quantitative RT-PCR was carried out according to the method described in Johmura et al., Molecular Cell 55: 73-84, 2014. Total RNA was extracted using ISOGEN II (Wako), and using this as a template, cDNA was synthesized using SuperScript II cDNA synthesis kit (Invitrogen). PCR amplification was carried out in a 96-well optical reaction plate, using Power SYBR Green PCR Master Mix (Applied Biosystems). The relative expression ratio of individual genes was normalized with respect to the expression level of GAPDH.

### 1-8. Ubiquitination Assay

In order to detect ubiquitinated KDM4B in vivo, the cells were transfected with a plasmid comprising a 2× Strep II (WSHPQFEKGGGSGGGSGGSAWSHPQFEK: SEQ ID NO: 8)-tagged KDM4B sequence, and were then treated with 20 μIVI MG132 for 16 hours. Forty-eight hours after the transfection, the cells were recovered. The cells were dissolved in a buffer containing 1% SDS under denaturation conditions, and after completion of centrifugation, a supernatant was diluted according to the methods described in Nishikawa et al., The Journal of biological chemistry 279: 3916-3924, 2004 and Sato et al., The Journal of biological chemistry 279: 30919-30922, 2004. A pulldown assay with 10 μl of a 50% StrepTactin resin was carried out using a buffer with a high salt concentration (2M NaCl, 50 mM Tris-HCl pH 7.5, 0.5% Nonidet P-40, 150 mM NaCl, 50 mM NaF, and 1 mM dithiothreitol). The resin was boiled in a sample buffer, and was then used in immunoblotting.

### 1-9. Fluorescence Microscope

U205-LacO-I-SceI-TetO cells were cultured in a glass bottom dish (Iwaki) on the stage of BZ-9000 (Keyence) equipped with an environmental chamber (Keyence). The microscopic image was analyzed with BZ-9000 software.

### 1-10. Gene Knockout by CRISPR/Cas9

An oligonucleotide of sgRNA for knocking-out human Fbxo22 was prepared, and was then cloned into the Bbsl site of the vector pX330 (obtained from Dr. Feng Zhang) expressing Cas9 and sgRNA. The obtained plasmid (pX330-hFbxo22-4) was transfected into MCF-7 cells or T47D cells, using Lipofectamine 3000 (Invitrogen). After the cells had been incubated for 48 hours, the cells were sub-cultured for cloning. The cell lysate of each cell line was used in Western blotting using an anti-Fbxo22 antibody, and whether the gene was destructed was confirmed. The sgRNA sequence of human Fbxo22 is 5′-CGCCGGAACCAGTCCTACGG-3′ (SEQ ID NO: 9).

### 1-11. ChIP-Seq Analysis

Chromatin immunoprecipitation was carried out using SimpleChIP Enzymatic Chromatin IP kit (CST).

4×10⁶ cells were fixed with 1% formaldehyde at room temperature for 10 minutes, and thereafter, 125 mM glycine was added to the cells. Chromatin was prepared from cell pellets, and was then treated with micrococcal nuclease at room temperature for 15 minutes. The cleaved chromatin and approximately 2 μg of each type of antibody were incubated at 4° C. overnight. Thereafter, 20 μl of magnetic beads were added thereto, and the obtained mixture was then incubated at 4° C. for 2 hours. The magnetic beads were washed with a washing buffer four times, and the chromatin was then eluted with a ChIP elution buffer, which was then treated with Protein K at 65° C. for 4 hours to perform reverse crosslink. Thereafter, DNA was extracted using a DNA purification column. A sequencing library was prepared with 1.8 ng of DNA, using Ion Xpress Plus Fragment Library kit (Thermo Fisher Scientific). Sequencing was carried out using Ion PI Chip and Ion PI Sequencing 200 kit, according to Ion Proton system (Thermo Fisher Scientific). The base-call and alignment in a single-ended read operation were carried out under default setting, using Torrent Suite™ Software 5.2.2. The read was mapped with respect to the human genome hg19 used as a control according to Torrent Mapping Alignment Program (TMAP). A tag directory was produced from the aligned read, using makeTagDirectory version v4.9.1 provided by HOMER (http://homer.salk.edu/homer/). Thereafter, using Homer, basic quality control analysis and sequence bias analysis were carried out. Using a default parameter, namely, a parameter having a tag density that is 4 or more times the normalized tag density in a region 10 kb from each peak, and a false discovery rate of 0.001 or less, the peak was called using rfindPeaks. In order to find a concentrated TF motif, findMotifsGenome was used at a region size of 50 bp and at a p-value of less than 1e-50, so that the region was annotated with annotatePeaks. The peak of the overlapped ER and SRC-3 was identified with intervals of less than 100 bp between the concentrated ER and SRC-3 regions. The hierarchical cluster analysis of the concentrated region was carried out using Cluster 3.0. Heat map data matrix was produced using Homer, and was then visualized using Java TreeView. A box-and-whisker plot was produced using Excel by taking log 2-ratio of each density.

1-12. Transplantation of Cancer Cells into Mice

Control (wild-type) T47D cells or Fbxo22-KO (knockout) T47D cells were cultured until the cells became 80% to 90% confluent. Thereafter, the cells were treated with trypsin, were then suspended in PBS, and were then mixed with Matrigel (CORNING 354230) at a mixing ratio of 1:1. An estrogen pellet (a 60-day slow-release pellet containing 0.72 mg of estrogen (Innovative Research of America)) was embedded into the subcutis of the nape of a mouse, and one day after the embedding, the cells (3×10⁶ cells) were injected together with 100 μl of PBS/Matrigel (1:1) into the subcutis of the mammary gland fat pad of a 9-week-old NOD/Scid mouse. When the size of a tumor became approximately 50 mm³, a tamoxifen pellet (a 60-day slow-release pellet containing 5 mg of tamoxifen (Innovative Research of America)) was embedded into the subcutis of five mice in each group. The size of the transplanted tumor was measured every week. Six weeks later, the mice were euthanized, the tumor was then excised, and the size of the tumor was then measured. Thereafter, the tumor was fixed with formalin and was then embedded in paraffin, and an HE stain slide glass was produced. The size of a tumor was evaluated as follows: V=(length×width×height×0.5326) mm³. All of the mice were fed under a specific pathogen-free environment, and were treated in accordance with Animal Experiment Guidelines, Graduate School of Medicine and Faculty of Medicine, The University of Tokyo.

### 1-13. Immunohistochemical Analysis of Tumor Section

The transplanted tumor tissues were fixed with 10% formalin, were then dehydrated, and were then embedded in paraffin. The paraffin section was deparaffinized, was then rehydrated, and was then incubated together with an anti-Ki-67 antibody (DAKO) or an antibody reacting against cleaved caspase-3 (CST). A primary antibody bound to the antigen was detected with an HRP-labeled secondary antibody, and was then visualized with DAB (3,3′-diaminobenzidine tetrahydrochloride).

### 1-14. Immunohistochemical Analysis of Patient-Derived Tissue Specimen

Formalin fixing, paraffin embedding, core needle aspiration biopsy cytology panels, and the clinical data thereof, which were derived from 163 cases of continuously treated T2 (diameter: 2 to 5 cm) ERα-positive/HER-2-negative primary breast cancers, were obtained from patients who underwent surgery at St. Marianna University School of Medicine Hospital from 2005 to 2009. The median follow-up time was 7.4 years. The present research was approved by the clinical trial review committee of the university (approval number: 3095). The immunohistochemical analysis was carried out using Nichirei Histofine system (Nichirei Biosiences Inc., Tokyo, Japan). A tissue section was incubated with anti-Fbxo22 antibody (GTX117774, GeneTex) used as a primary antibody, which was diluted to 1:200, and was then detected using an HRP-polymer-labeled secondary antibody (Histofine Simple Stain MAX PO (multi), Nichirei, Japan). Coloration was carried out using DAB. Cancer tissues comprising one or more cells exhibiting Fbox22 staining at a moderate degree or a high degree (i.e. Fbxo22-positive cells) out of 100 cells were determined to be Fbxo22-positive. The Fbxo22-positive cells were evaluated according to a blind test conducted by two pathologist. The statuses of ERα, PR, HER2, and Ki-67 were determined by a standard immunohistochemical method and a FISH (fluorescence in situ hybridization) method, which are used in clinical studies.

### 1-15. Statistical Analysis

The statistical analysis of a cell-based experiment was carried out according to a Student's t-test to an independent variable. P value<0.05 was defined to be statistically significant. A statistical analysis regarding tag counting of ChIP-seq data was carried out by one-way ANOVA. The relationship between Fbxo22 status and various clinicopathological features was calculated by a chi-square test, a Fisher's exact test, and a Student's t-test. A relapse-free survival (RFS) curve was produced by a Kaplan-Meier method and a log-rank test. Regarding individual variables in a single variable analysis and a multivariate analysis, a cox proportional hazard regression model was used to evaluate the hazard ratio (HR) and 95% confidence intervals (95% CIs) of RFS. A Kaplan-Meier plot was produced using GraphPad Prism6. The cox proportional hazard regression model was analyzed by R (version 3.3.2). The statistical significance was defined as P<0.05.

### 2. Results

### 2-1. Breast Cancer

### 2-1-1. KDM4B Ubiquitination and Influence of its Decomposition on Agonistic Activity of TAM

If E2-induced transcriptional activity in MCF-7 cells is regulated by proteasome-dependent decomposition, the antagonistic activity of TAM is also likely to be regulated by proteasome-dependent proteolysis.

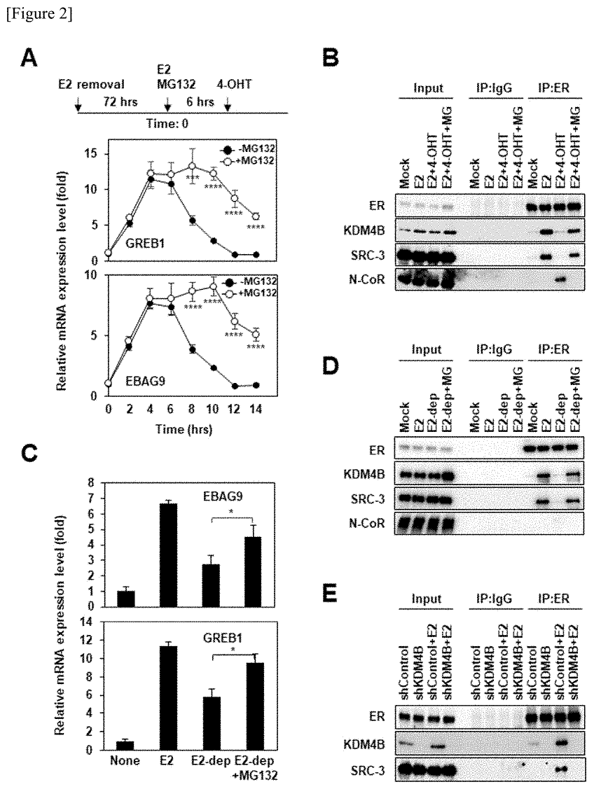

In order to examine this assumption, first, the kinetics of the antagonistic activity of 4-hydroxytamoxifen (4-OHT) were measured. E2 was added to MCF-7 cells in an E2-depleted condition to stimulate the cells, and 6 hours after the addition of E2, 4-OHT was added to the cells. The transcript levels of GREB1 and EBAG1 that were target genes of ERα were measured over time. As a result, 4 hours after the addition of E2, the transcript levels became maximum, and were then reduced. Six hours after the addition of 4-OHT, the transcript levels became minimum (FIG. 2A, ●). From these results, it was demonstrated that estrogen signals are promptly antagonized after the treatment with TAM. Thus, whether or not MG132 serving as a proteasome inhibitor influences on the antagonizing activity of ERα-dependent transcription by 4-OHT was examined. After the treatment with 4-OHT, the transcript levels of EBAG9 and GREB1 were maintained at high levels in the presence of MG132, compared with in the absence of MG132 (FIG. 2A).

After the treatment with E2, ERα forms a complex with KDM4B and SRC-3 (FIG. 2B, E2 of IP:ER). This complex was dissociated by addition of 4-OHT, and ERα formed a repressor with an N-CoR repressor (FIG. 2B, E2+4-OHT of IP:ER). By treating with MG132, the dynamics of these cofactors were suppressed (FIG. 2B, E2+4-OHT+MG of IP:ER). The same results were obtained also in the case of using T47D cells.

In addition, when E2 was removed from the medium containing E2, the E2-induced transcriptional activity of EBAG9 and GREB1 disappeared (FIG. 2C), and when MG13 was added, this E2-induced transcriptional activity was recovered. Moreover, dissociation of KDM4B and SRC-3 from ERα by elimination of E2 was suppressed (FIG. 2D, E2+dep+MG of IP:ER). From these results, it was likely that the decomposition by proteasome of KDM4B that forms a complex with ERα would trigger the dynamics of ERα cofactors. Hence, the expression of KDM4B in the cells was inhibited by shRNA. As a result, even in the presence of E2, SRC-3 was dissociated from ERα (FIG. 2E, shKDM4B+E2 of IP:ER).

From the aforementioned results, it is considered that the dynamics of cofactors are triggered by the decomposition by proteasome of KDM4B that binds to ERα, and that the antagonistic activity of TAM in ERα-positive breast cancer cells is promoted.

2-1-2. Identification of Factor Associated with Decomposition of KDM4B

Next, a factor that selectively decomposes KDM4B forming a complex with ERα was searched.

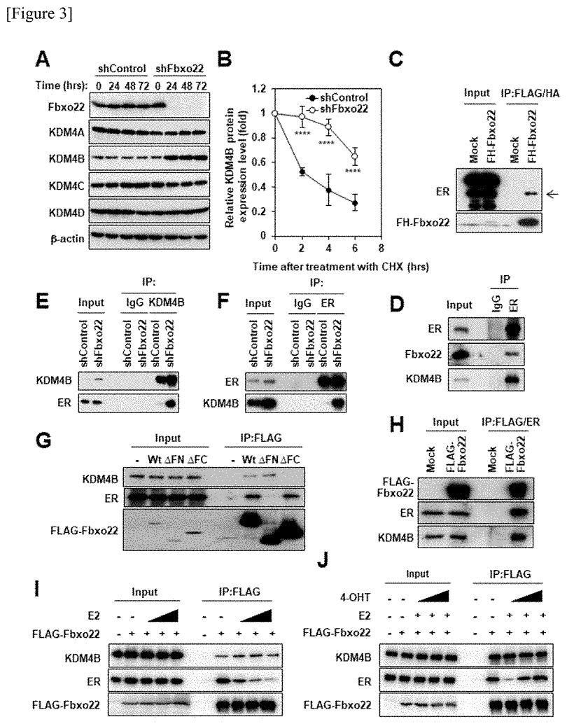

It has been reported that Fbxo22 is somewhat correlated with the function of KDM4A. Thus, whether or not Fbxo22 is associated with decomposition of KDM4B was examined. The steady-state levels of KDM4A, 4C and 4D in Fbxo22-depleted cells were equivalent to the steady-state level of wild-type cells, but the amount of KDM4B was apparently increased (FIG. 3A, KDM4B of shFbxo22). Besides, the mRNA level of KDM4B was not fluctuated. In the Fbxo22-depleted cells, the KDM4B protein was present more stably than in control cells (FIG. 3B, 0).

In order to examine whether or not SCF^{Fbxo22} ubiquitinates KDM4B that forms a complex with ERα, first, formation of a complex between ERα and Fbxo22 was examined. FLAG-HA-tagged Fbxo22 (FH-Fbxo22) was allowed to express in MCF-7 cells in the presence of MG132, and immunoprecipitation was carried out using an anti-FLAG antibody and an anti-HA antibody. As a result, ERα was contained in the immunoprecipitated obtained with the anti-FLAG/anti-HA antibodies. Hence, it was found that ERα interacts with Fbxo22 (FIG. 3C, FH-Fbxo22 of IP: FLAG/HA). Then, when Fbxo22 was eliminated in the absence of MG132, the interaction between ERα and KDM4B was significantly promoted (FIG. 3E, shFbxo22 of IP:KDM4B; FIG. 3F, shFbxo22 of IP:ER). Since Fbxo22 has three different functional domains (F-box, FIST-N, and FIST-C), it was considered that Fbxo22 forms a multimer with ERα. As such, using an Fbxo22 mutant deleting a FIST-N or FIST-C domain, the interaction between ERα and KDM4B was examined. As a result, it became clear that ERα and KDM4B bind to a FIST-N domain and a FIST-C domain, respectively (FIG. 3G). Furthermore, when FLAG-Fbxo22 was allowed to express in MCF-7 cells in the presence of MG132, FLAG-Fbxo22, ERα, and KDM4B were contained in the immunoprecipitate obtained with an anti-FLAG antibody and an anti-ERα antibody. From these results, it was found that these three molecules form a complex (FIG. 3H).

Subsequently, the interaction of an agonist or an antagonist binding to ERα with Fbxo22 was examined. In the presence of MG132, FLAG-Fbxo22 binding to ERα was reduced dependently on the additive amount of E2, and the binding to KDM4B was not influenced (FIG. 3I). In contrast, in the presence of E2 and MG132, FLAG-Fbxo22 binding to ERα was recovered (increased) dependently on the additive amount of 4-OHT (FIG. 3J). These results demonstrate that Fbxo22 preferentially binds to ligand-non-bound ERα or 4-OHT-bound ERα, and does not bind to E2-bound ERα.

### 2-1-3. Ubiquitination of KDM4B by SCF^{Fbxo22} (SCF-Fbxo22 Complex)

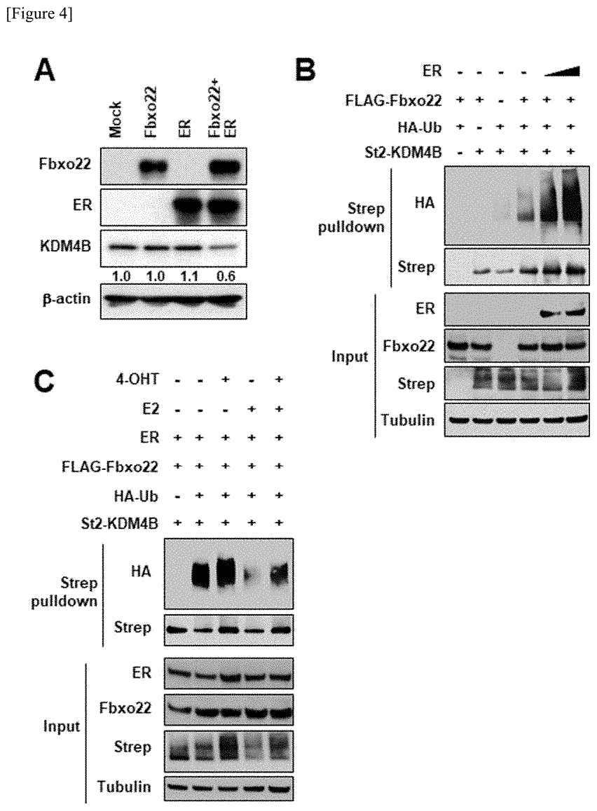

In order to examine the influence of Fbxo22 or ERα on the amount of KDM4B in MCF-7 cells, Fbxo22 and/or ERα were allowed to express in MCF-7 cells. As a result, when Fbxo22 or ERα was allowed to express alone, it did not influence on the amount of KDM4B. However, when Fbxo22 and ERα were allowed to simultaneously express, the amount of KDM4B was reduced (FIG. 4A). These results suggest that SCF^{Fbxo22} specifically ubiquitinates KDM4B that forms a complex with ERα.

In order to further examine this possibility, an in vivo ubiquitination assay was carried out under denaturation conditions. Ubiquitination of KDM4B by Fbxo22 was weak when ERα was not expressed. However, when ERα was co-expressed, ubiquitination of KDM4B was significantly reinforced in an ERα dose dependent manner (FIG. 4B). These results suggested that SCF^{Fbxo22} preferentially ubiquitinates KDM4B forming a complex with ERα and causes decomposition by proteasome.

If Fbxo22 preferentially binds to ligand-non-bound ERα or 4-OHT-bound ERα, the ubiquitination by SCF^{Fbxo22} of KDM4B forming a complex with ERα is considered to depend on the type of a ligand binding to Ra. Ubiquitination of KDM4B forming a complex with ERα was significantly reduced by addition of E2, and when 4-OHT was added, the amount of ubiquitination was recovered (FIG. 4C).

From the aforementioned results, it was demonstrated that SCF^{Fbxo22} preferentially ubiquitinates KDM4B forming a complex with ligand-non-bound ERα or 4-OHT-bound ERα.

### 2-1-4. Influence of Decomposition of KDM4B Mediated by SCF^{Fbxo22} on Antagonistic Activity of SERM

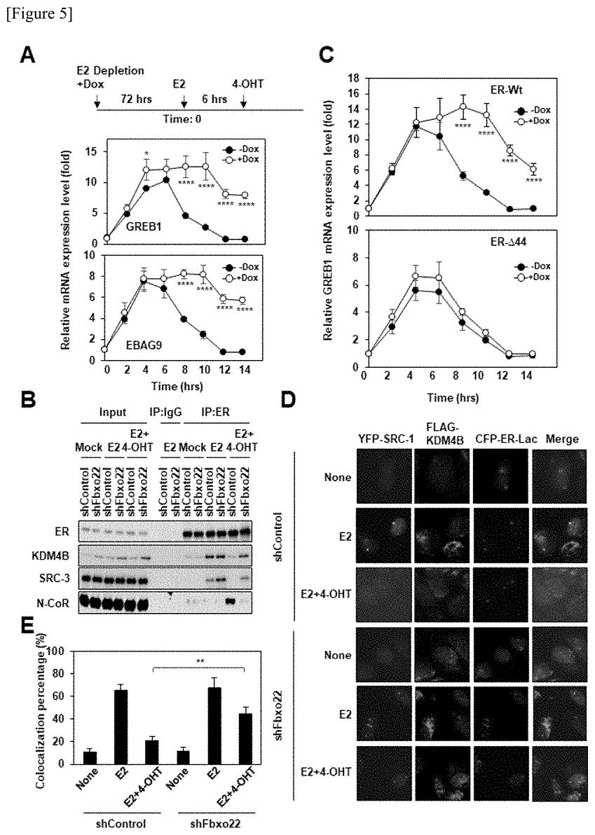

When control MCF-7 cells stimulated by E2 were treated with 4-OHT, transcription of EBAG9 and GREB1 was strongly suppressed (FIG. 5A, ●), and this antagonistic activity of 4-OHT was suppressed in Fbxo22-depleted MCF-7 cells (FIG. 5A, ◯). After the stimulation by E2, ERα formed a complex with KDM4B and SRC-3 in both the control MCF-7 cells and the Fbxo22-depleted MCF-7 cells (FIG. 5B, IP:E2). Thereafter, when the control MCF-7 cells were treated with 4-OHT, KDM4B and SRC-3 were dissociated from ERα, and N-CoR interacted with ERα (FIG. 5B, shFbxo22 of IP:E2). In contrast, in the Fbxo22-depleted cells, the dynamics of such cofactors were not observed (FIG. 5B, shControl of IP:E2). In addition, when KDM4B was removed, induction of the transcription of the ERα target genes was significantly suppressed regardless of the presence of Fbxo22. Even by removing KDM4B, induction of the transcription of EBAG9 and other ERα target genes by E2 was significantly suppressed. Thus, association of KDM4B with the control of the antagonistic activity of 4-OHT was also intended to be confirmed.

The transcriptional activity of ERα is caused by AF1 and AF2. Hence, whether or not ERα activity in Fbxo22-depleted cells in the presence of 4-OHT depends on AF1 and/or AF2 was examined.

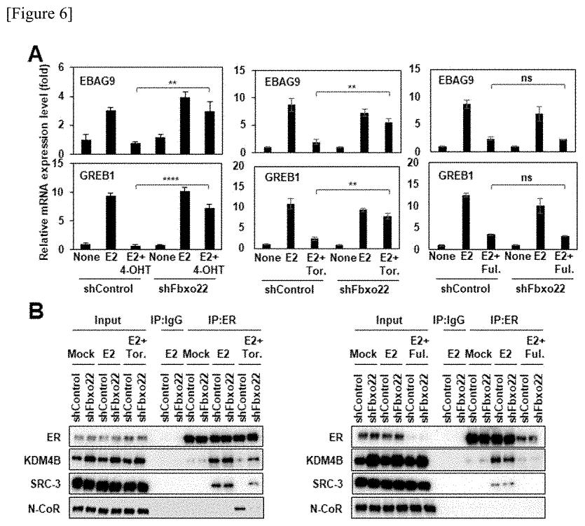

Fbxo22-expressing U2OS cells or Fbxo22-non-expressing U2OS cells, which express the entire-length wild-type ERα or Δ44 mutant ERα deleting AF1 activity, were stimulated by E2 and were then treated with 4-OHT. The antagonistic activity of 4-OHT was suppressed in U2OS cells, in which Fbxo22 was not expressed but wild-type ERα was expressed, and which were stimulated by E2 (FIG. 5C, upper view, ◯). On the other hand, in U2OS cells, in which Fbxo22 was not expressed but Δ44 ERα was expressed, the antagonistic activity of 4-OHT was observed (FIG. 5C, lower view, ◯). These results show that ERα activity in Fbxo22-depleted cells in the presence of 4-OHT depends on AF1 activity. The role of Fbxo22 in the antagonistic activity on ERα signals was not specific to SERD (selective estrogen receptor down-regulator) but was specific to SERM (selective estrogen receptor modulator). By removing Fbxo22, effects similar to those on 4-OHT were brought on the antagonistic activity of Toremifene (SERM) (FIG. 6A, left view and center view), but no effects were brought on the antagonistic activity of Fulvestrant (SERD) (FIG. 6A, right view). Moreover, Fbxo22 was necessary for the dynamics of cofactors in Toremifene-treated cells (FIG. 6B, left view, E2+Tor of IP:ER), but it was not necessary in Fulvestrant-treated cells (FIG. 6, right view, E2+Ful of IP:ER). Accordingly, these results demonstrate that decomposition of ERα-bound KDM4B by SCF^{Fbxo22} was necessary for cofactor dynamics specific to the SERM treatment.

In order to further analyze the role of Fbxo22 in accumulation of a coactivator and ERα induced by a ligand, the intracellular dynamics of an ERα-coactivator complex in living cells were observed in real time. In order to immobilize ERα on a Lac operator array stably incorporated into living cells, using a CFP-tagged lac receptor-ERα chimeric protein (CFP-LacER), accumulation of YFP-SRC-1 and KDM4B in an ERα-immobilized position was examined.

First, with regard to colocalization of FLAG-KDM4B in CFP-LacER and YFP-SRC-1 foci, an analysis was carried out using U2OS-LacO-I-SceI TetO cells expressing CFP-LacER, YFP-SRC-1 and FLAG-KDM4B. According to an immunofluorescence analysis using an anti-FLAG antibody, in the presence of E2, FLAG-KDM4B was also colocalized in CFP-LacER and YFP-SRC-1 foci in control cells and Fbxo22-depleted cells (FIG. 5D, E2 of shControl and shFbxo22; FIG. 5E, E2 of shControl and shFbxo22). In contrast, in the presence of E2 and 4-OHT, colocalization of FLAG-KDM4B with CFP-LacER and YFP-SRC-1 was not observed in control cells (FIG. 5D, E2+4-OHT of shControl; FIG. 5E, E2+4-OHT of shControl), whereas FLAG-KDM4B colocalized with CFP-LacER and YFP-SRC-1 in Fbxo22-depleted cells (FIG. 5D, E2+4-OHT of shFbxo22; FIG. 5E, E2+4-OHT of shFbxo22).

From the aforementioned results, it was demonstrated that Fbxo22 plays an important role in cofactor dynamics in the action of 4-OHT to antagonize E2 signaling.

2-1-5. Role of Fbxo22 in Dissociation of 4-OHT-Dependent SRC-3 from ERα-SRC3-Binding Genomic Region

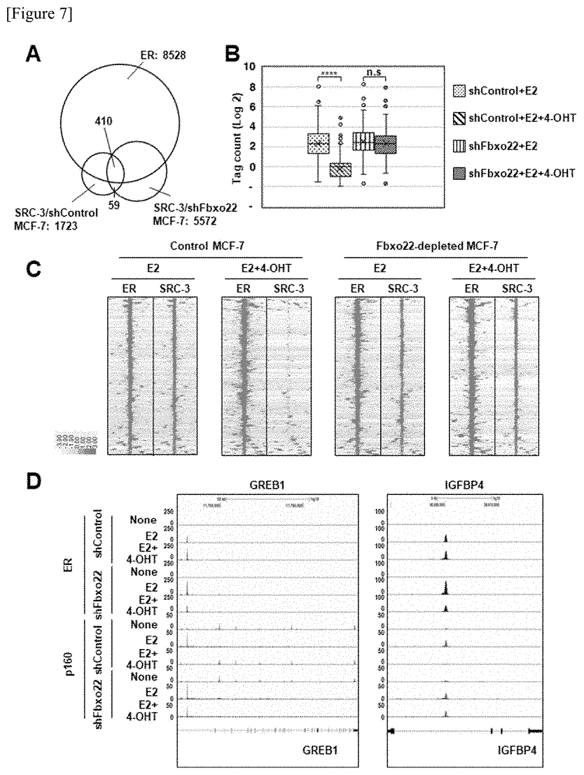

Applying a ChIP-Seq analysis, the genome-wide binding of ERα-SRC-3 was mapped. The analysis was carried out using control MCF-7 cells and Fbxo22-depleted MCF-7 cells. The cells were left under an estrogen-depleted condition for 72 hours, and were then stimulated by E2 (10 nM) or E2+4-OHT for 2 hours. As a result, 1,264 ERα accumulation peaks were detected in E2-treated wild-type MCF-7 cells, and 23,213 ERα accumulation peaks were detected in Fbxo22-depleted MCF-7 cells. In addition, 26,751 ERα accumulation peaks were detected in E2+4-OHT-treated control MCF-7 cells, and 19,924 ERα accumulation peaks were detected in Fbxo22-depleted MCF-7 cells. From these results, it is considered that 4-OHT does not affect the interaction of ERα with a target region. In the above-described 4 data sets, 8,528 ERα accumulation peaks were overlapped. In E2-treated wild-type MCF-7 cells, 1,723 SRC-3 accumulation peaks were detected. In Fbxo22-depleted MCF-7 cells, 5,572 SRC-3 accumulation peaks were detected. Between these two data, 469 SRC-3 accumulation peaks were common, and approximately 90% (410) of these peaks were overlapped with ERα peaks (FIG. 7A). In order to reveal the amount of SRC-3 interacting with ERα, 410 SRC-3 overlapping peaks were focused. As a result of a box-and-whisker plot, it was found that the amount of SRC-3 interacting with ERα was significantly reduced in E2+4-OHT-treated control MCF-7 cells (FIG. 7B, shControl+E2+4-OHT), whereas the amount of SRC-3 interacting with ERα was not reduced in Fbxo22-depleted MCF-7 cells (FIG. 7B, shFbxo22+E2+4-OHT). In a heat map, in E2+4-OHT-treated control MCF-7 cells, the read density of the SRC-3 sequence in 410 regions was apparently reduced, compared with the case of treating with only E2 (FIG. 7C, Control MCF-7, E2+4-OHT). In contrast, even though E2+4-OHT was added to Fbxo22-depleted MCF-7 cells, it did not affect the read density of the SRC-3 sequence (FIG. 7C, Fbxo22-depleted MCF-7, E2+4-OHT).

In fact, when control MCF-7 cells and Fbxo22-depleted MCF-7 cells were treated with E2, accumulation of ERα in the promoter regions of GREB1 and IGFBP4 genes was promoted (FIG. 7D). Accumulation of ERα in the promotors was maintained even after the treatment with E2+4-OHT in both the control MCF-7 cells and the Fbxo22-depleted MCF-7 cells. In contrast, in the case of SRC-3 (p160), SRC-3 was dissociated from the promoter after the treatment with E2+4-OHT in the control MCF-7 cells, but SRC-3 remained in the promoter even after the treatment with E2+4-OHT in the Fbxo22-depleted MCF-7 cells.

From these results, it was demonstrated that Fbxo22 plays an important and universal role in the dissociation of SRC-3 from the ERα/SRC-3-binding genomic region, which is induced by tamoxifen.

### 2-1-6. Influence of Fbxo22 on Breast Cancer Cell Proliferation Inhibitory Activity of TAM In Vitro and In Vivo

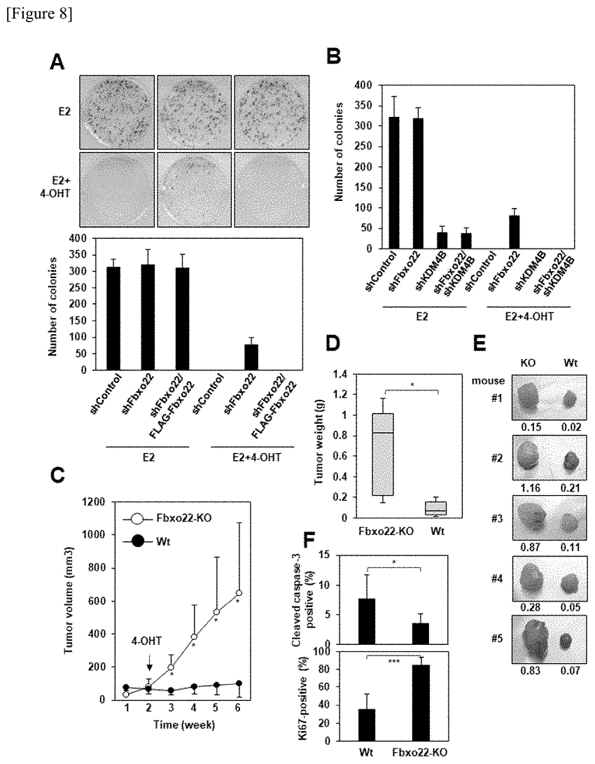

Estrogen is necessary for proliferation of MCF-7 cells. Thus, whether or not Fbxo22 is necessary for the suppression of proliferation of MCF-7 cells by 4-OHT was examined. As a result of a colony formation assay, it was found that proliferation of control MCF-7 cells in the presence of E2 was completely suppressed by being treated with 4-OHT, and that the proliferation-suppressing effect of 4-OHT was reduced in Fbxo22-depleted MCF-7 cells (FIG. 8A, lower view, shFbxo22 of E2+4-OHT), but when Fbxo22 was allowed to express in the cells, the proliferation-suppressing effect was recovered (FIG. 8A, lower view, shFbxo22/FLAG-Fbxo22 of E2+4-OHT). The same results were observed even in two other types of breast cancer cell lines (ZR75-1 and T47D). It has been known that proliferation of these two types of breast cancer cell lines depends on estrogen stimulation. Even in a case where KDM4B was removed, proliferation of MCF-7 cells induced by E2 was suppressed regardless of the presence of Fbxo22 (FIG. 8B).

Subsequently, whether or not Fbxo22 is also necessary for the antagonistic activity of 4-OHT in vivo was examined. Control T47D breast cancer cells or Fbxo22-knocked-out T47D breast cancer cells (3×10⁶ cells) were transplanted into the mammary gland fat pad of female NOD/Scid mice. Two weeks after the transplantation, 4-OHT pellets were transplanted into the mice, and the degree of proliferation of a tumor was then examined. Before the 4-OHT treatment, both the control cells and the Fbxo22-knocked-out cells formed a tumor having almost the same size in all of the transplanted mice, and thus, it was confirmed that the removal of Fbxo22 does not affect proliferation of T47D cells in the mammary gland fat pad. However, unexpectedly, when the mice into which Fbxo22-knocked-out cells had been transplanted were treated with 4-OHT, a clear increase in the tumor was observed, compared with the mice into which control cells had been transplanted (FIG. 8C). Being consistent with these results, colony formation of the Fbxo22-knocked-out T47D cells was significantly promoted even in the presence of E2 and 4-OHT, compared with the control cells.

Six weeks after the transplantation, the mice were euthanized, and the weight of the tumor, etc. was then measured. The tumor of Fbxo22-knocked-out T47D cells was apparently heavier than the tumor of wild-type T47D cells (FIG. 8D). FIG. 8E shows comparative examples of representative tumors. When an immunohistochemical analysis was carried out on tumor tissues, apoptosis was reduced in Fbxo22-knocked-out T47D cells (FIG. 8F, upper view), and cell proliferation was increased (FIG. 8F, lower view).

From the aforementioned results, it was demonstrated that Fbxo22 is essential for the antagonistic activity of 4-OHT, in vivo and in vitro, through decomposition of KDM4B that forms a complex with ERα.

### 2-1-7. Significance of Expression Level of Fbxo22 in Determination of Prognosis of ERα-Positive/HER2-Negative Breast Cancer Patients

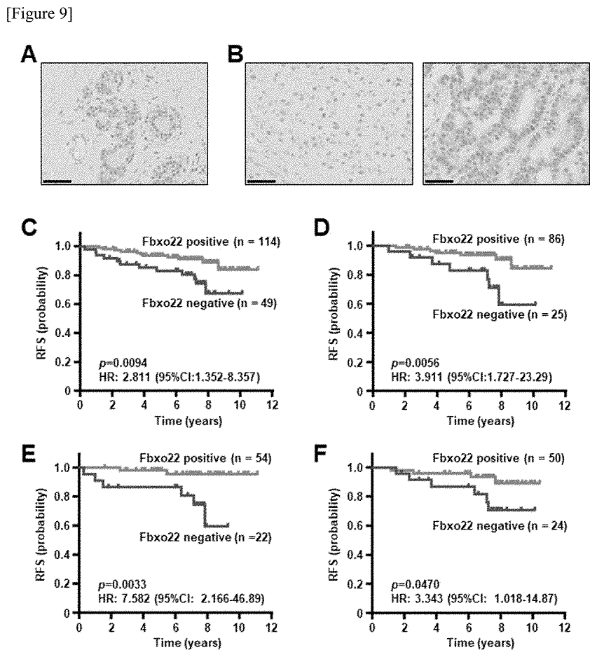

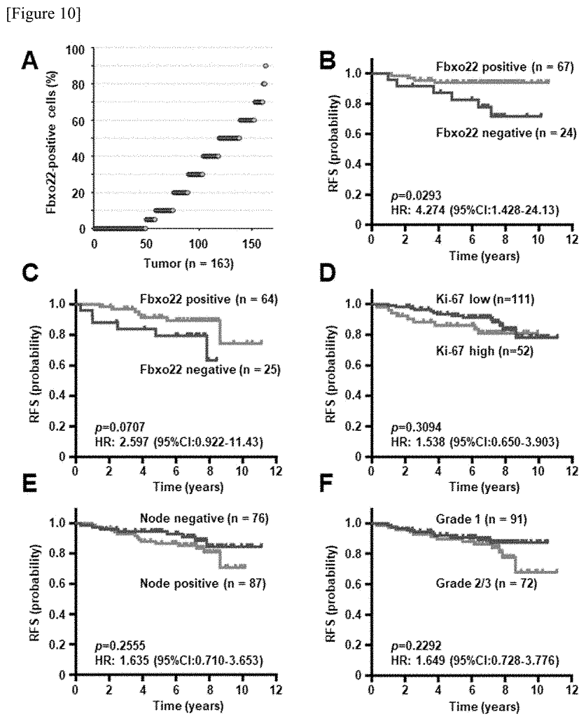

The important role of SCF^{Fbxo22} as a TAM sensitivity determining factor in breast cancer cells suggests that the expression level of Fbxo22 can be used as a prognosis-determining factor for patients with ERα-positive breast cancer. Hence, in order to confirm the possibility of the expression level of Fbxo22 as a prognosis-determining factor for patients with ERα-positive breast cancer, 163 ERα-positive/HER2-negative breast cancer specimens were analyzed in terms of the expression level of Fbxo22 according to an immunohistochemical method. When the specimens were immunostained using an anti-Fbxo22 antibody, the cell nucleus was unevenly stained in normal mammary gland-derived tissues (FIG. 9A). In contrast, in tumor tissues, both a case where the cell nucleus was evenly stained (FIG. 9B, left view) and a case where the cell nucleus was hardly stained (FIG. 9B, right view) were observed. Herein, with regard to the indicator of the expression level of Fbxo22 in cancer tissues, when the percentage of Fbxo22-positive cells whose nuclei were stained with the anti-Fbxo22 antibody (i.e., cells exhibiting Fbxo22 stainability at a moderate degree or a high degree) was less than 1%, the cancer tissues were defined to be Fbxo22-negative. As a result of the observation, 49 specimens (30.1%) out of 163 specimens were determined to be Fbxo22-negative (Table 3 and FIG. 10). The Fbxo22-negative expression was correlated with high Ki-67 and PR status negative (Table 3).

TABLE 3

| | Fbxo22 expression | | |
|---|---|---|---|
| Characteristics | positive | negative | P |
| Total samples | 114 | 49 | |
| age, years | | | |
| Mean | 56 | 54 | |
| SD | 13 | 13 | 0.4036 [symbol] |
| Node | | | |
| Positive | 60 | 27 | |
| Negative | 54 | 22 | 0.7719 [symbol] |
| Grade | | | |
| Low | 67 | 24 | |
| Medium/High | 47 | 25 | 0.2583 [symbol] |
| Histology [symbol] | | | |
| IC of NST (invasive ductal) | 95 | 35 | |
| Invasive lobular Ca | 4 | 7 | |
| Mucinous Ca | 8 | 2 | |
| Ca with apocrine differentiation | 3 | 0 | |
| Others | 4 | 5 | 0.02797 [symbol] |
| PR [symbol] | | | |
| Positive | 95 | 33 | |
| Negative | 19 | 16 | 0.0227 [symbol] |
| Ki67 [symbol] | | | |
| high | 28 | 24 | |
| low | 86 | 25 | 0.0022 [symbol] |
| Chemotherapy | | | |
| No | 52 | 17 | |
| Yes | 62 | 32 | 0.1957 [symbol] |
| Hormone therapy | | | |
| No | 3 | 2 | |
| tamoxifen | 32 | 14 | |
| aromatase inhibitors | 59 | 23 | |
| both (TAM & AI) | 18 | 10 | |
| Others | 2 | 0 | 0.8386 [symbol] |

¹⁾Histology was determined based on WHO 2012 classification IC of NST: invasive carcinoma of no special type
²⁾PR status: 0% vs 1%≤, Ki67 status: ≤ 10% vs 20%≤
³⁾unpaired t-test
⁴⁾Chi-square test
⁵⁾Fisher's exact test [symbol]
indicates data missing or illegible when filed

However, the Fbxo22 status was not correlated with lymph node metastasis and tumor grading (Table 3). It is particularly noteworthy that the Fbxo22-depleted tumor has a significant correlation with a reduction in the relapse-free survival (RFS), compared with the Fbxo22-positive tumor (FIG. 9C). This reduction in RFS was also observed in Luminal A-type (low Ki-67) breast cancer (FIG. 9D), lymph node metastasis-negative breast cancer (FIG. 9E), and grade 1 ERα-positive/HER2-negative breast cancer (FIG. 10B). Moreover, Fbxo22-negative breast cancer had an extremely poor prognosis in the group that had undergone the TAM treatment (FIG. 9F), but the prognosis of the Fbxo22-negative breast cancer was not so bad in the group that had not undergone the TAM treatment (FIG. 10C). In a total of 163 groups, the high Ki-67 group, the lymph node metastasis group, and the grade 2/3 group tended to have a poor prognosis, but there was no statistically significant difference among them (FIGS. 10D, E and F).

Furthermore, it was demonstrated according to a multivariable survival analysis that Fbxo22 depletion predicts prognosis, independently from other prognosis-determining factors (Table 4). These clinical data show that Fbxo22 depletion causes a poor prognosis to ERα-positive/HER2-negative breast cancer, irrelevantly to low Ki-67, negative lymphatic metastasis, low grade, or tamoxifen treatment.

TABLE 4

| | Univariate | | | Multivariate | | |
|---|---|---|---|---|---|---|
| Variable | HR | 95% CI | P | HR | 95% CI | P |
| Fbxo22 | | | | | | |
| Positive | 1.00 | | | 1.00 | | |
| Negative | 2.847 | 1.248 to 8.493 | 0.012 [symbol] | 2.6483 | 1.1148 to 6.291 | 0.0274 |
| Node | | | | | | |
| Negative | 1.00 | | | 1.00 | | |
| Positive | 1.639 | 0.5936 to 3.872 | 0.26 | 1.5679 | 0.6520 to 3.771 | 0.3151 |
| Grade | | | | | | |
| Low | 1.00 | | | 1.00 | | |
| Medium/High | 1.651 | 0.7231 to 3.768 | 0.234 | 1.483 [symbol] | 0.639 [symbol] to 3.442 | 0.3581 |
| PR | | | | | | |
| Negative | 1.00 | | | 1.00 | | |
| Positive | 0.7685 | 0.2983 to 1.929 | 0.562 | 0.8473 | 0.3257 to 2.204 | 0.7341 |
| Ki67 | | | | | | |
| Negative | 1.00 | | | 1.00 | | |
| Positive | 1.54 | 0.6856 to 3.582 | 0.313 | 1.0409 | 0.4293 to 2.524 | 0.92 [symbol] 3 |

[symbol]

Estimated from Cox proportional hazards model. [symbol]
indicates data missing or illegible when filed

### 2-2. Endometrial Cancer

Estrogen is associated with the onset of endometrial cancer. Endometrium, as well as bone, has been known as an organ, on which tamoxifen exhibits an agonistic action. Thus, the risk of developing endometrial cancer is increased by tamoxifen. As such, the agonistic action of tamoxifen is considered to cause an increase in the risk of endometrial cancer. That is, when tamoxifen is administered, the tamoxifen acts as an antagonist on Fbxo22-positive breast cancers, but it acts as an agonist on Fbxo22-negative breast cancers. From this fact and the aforementioned results regarding breast cancer, it is likely that Fbxo22-negative endometrial cancer is developed by administration of an anti-hormonal agent such as tamoxifen, and that the prognosis thereof becomes poor.

In order to analyze this, the expression of Fbxo22 in 30 cases of endometrial cancer (EC), 29 cases of atypical endometrial hyperplasia (AEH) that is a non-invasive early endometrial cancer, 30 cases of endometrial hyperplasia (EH) as a precancerous lesion, and 22 cases of normal endometrium, was analyzed by immunostaining using an Fbxo22 antibody (St. Marianna University School of Medicine, Bioethics committee; approval number: 4230). The expression degree was evaluated according to H-Score in the cell nucleus (i.e., an evaluation method commonly applied in pathological inspections, comprising evaluating with a total score of positive cell percentage x strongly positive 3 points, intermediately positive 2 points, and weakly positive 1 point). Ki-67 and the progesterone receptor (PgR) were evaluated with positive percentage (%) used in clinical sites.

### 2-2-1. Expression of Fbxo22 in Normal Endometrium

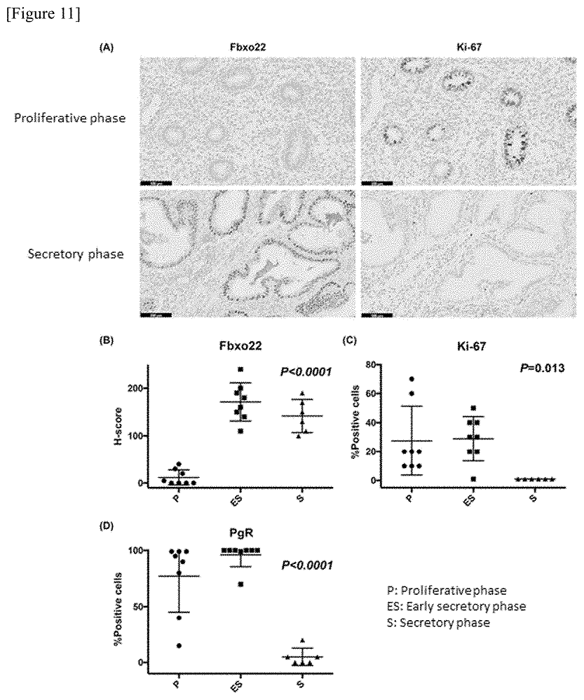

In a normal endometrium, the expression of Fbxo22 was changed depending on the menstrual cycle, and the expression was negative in the proliferative phase and was positive in the secretory phase (FIGS. 11A and B). The normal endometrium was divided according to morphological evaluation involving HE staining into 8 cases of endometrium proliferative phase (P) and 14 cases of secretory phase (S) (8 out of the 14 cases were divided into early secretory phase (ES)). In the proliferative phase, in all of the cases, H-Score was 40 or less and the mean value was 11.9±16.0. In contrast, in the early secretory phase, the mean value was the highest (171.3±40.2), and in other secretory phases, the mean value was 141.7±34.9. Ki-67 used as an indicator of proliferation was high in the proliferative phase and the early secretory phase, and it almost disappeared after the middle secretory phase (FIGS. 11A and C). Likewise, PgR was also high in the proliferative phase and the early secretory phase, and it was a low value after the middle secretory phase (FIG. 11D). From these findings, it was suggested that the expression of Fbxo22 is induced in concurrence with the secretory phase of a progesterone called LH surge in the menstrual cycle. In addition, it was also suggested that the expression of Fbxo22 tends to be inversely correlated with Ki-67, and thus that proliferation is induced if Fbxo22 is decreased. In all of the cases, ER is strongly positive, and it is theoretically predicted that continuous ER signals are generated as a result that Fbxo22 is not expressed.

### 2-2-2. Expression of Fbxo22 in Endometrial Cancer

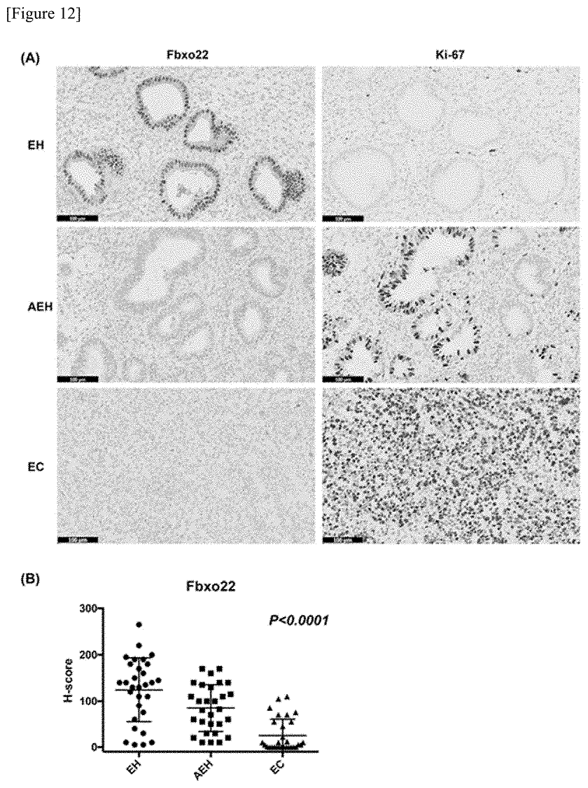

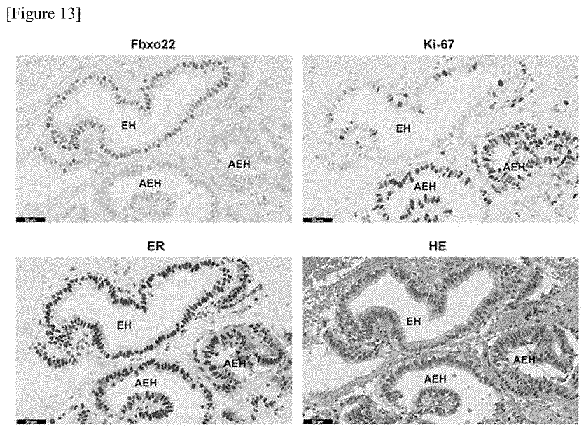

Regarding correlation with canceration, as expected, Fbxo22 was decreased in EC, and if the correlation is evaluated using H-Score, the score was gradually decreased such as EH: 124.2±69.1, AEH: 84.7±51.0, and EC: 25.0±35.3 (FIGS. 12A and B). In these cases, the expression of Fbxo22 tended to be inversely correlated with the expression of Ki-67 (FIG. 12A), whereas in the AEH cases in which both EH and AEH were present, Fbxo22 was positive in EH and was negative in AEH. Thus, an inversely correlated relationship was observed, such that Ki-67 was high in the Fbxo22-negative gland duct (FIG. 13).

As mentioned above, taking into consideration the fact that ER signals continue even if estrogen is depleted in an ERα-positive breast cancer model, it is likely that if Fbxo22 is decreased, introduction into the secretory phase caused by progesterone signals cannot be carried out smoothly, and that cell proliferation excessively occurs due to estrogen signals, thereby inducing cancer. As a clinical application, the expression of Fbxo22 is likely to become a predictive factor capable of predicting progression to AEH and EC in EH cases.

### INDUSTRIAL APPLICABILITY

The present invention relates to a method for determining the prognosis of a hormone receptor cancer and a method for evaluating therapeutic effects. Therefore, it is expected that the present invention will be utilized in the medical field.

1. A method for evaluating the prognosis of a hormone receptor-positive cancer, or assisting the evaluating thereof, the method comprising the following steps (a) and (b):
(a) a step of detecting Fbxo22-positive cancer cells in a sample derived from cancer tissues; and
(b) a step of calculating the percentage of the Fbxo22-positive cancer cells to cancer cells present in the sample.

2. The method according to claim 1, wherein the cancer is evaluated to have a poor prognosis, when the percentage of the Fbxo22-positive cancer cells is less than 10%.

3. The method according to claim 1, wherein the Fbxo22-positive cancer cells are detected by an immunohistochemical staining method.

4. The method according to claim 3, the Fbxo22-positive cancer cells are evaluated based on the stainability of the cells by an anti-Fbxo22 antibody.

5. The method according to claim 4, wherein the cells are evaluated to be Fbxo22-positive cancer cells, when the stainability is a moderate level or higher.

6. The method according to claim 1, wherein the hormone receptor-positive cancer is either an ERα (estrogen receptor α)-positive cancer or an AR (androgen receptor)-positive cancer.

7. The method according to claim 6, wherein the ERα-positive cancer is an ERα-positive breast cancer or an ERα-positive endometrial cancer.

8. The method according to claim 6, wherein the AR-positive cancer is an AR-positive prostate cancer.

9. (canceled)

10. The method according to claim 16, wherein the hormone receptor-positive cancer is either an ERα-positive cancer or an AR-positive cancer.

11. The method according to claim 10, wherein the ERα-positive cancer is an ERα-positive breast cancer or an ERα-positive endometrial cancer.

12. The method according to claim 11, wherein the anti-hormonal agent is SERM (selective estrogen receptor modulator).

13. The method according to claim 10, wherein the AR-positive cancer is an AR-positive prostate cancer.

14. The method according to claim 13, wherein the anti-hormonal agent is SARM (selective androgen receptor modulator).

15. A kit for evaluating the prognosis of a hormone receptor-positive cancer, or for evaluating the therapeutic effects of an anti-hormonal agent on the cancer, wherein the kit comprises an element for detecting the expression level of Fbxo22.

16. A method of treatment of a hormone receptor-positive cancer, comprising:
administering a therapeutically-effective amount of an anti-hormonal agent to a subject in need thereof, Fbxo22-positive cancer cells having been detected in a sample derived from cancer tissue from the subject, the percentage of the Fbxo-22-positive cancer cells to cancer cells having been calculated to be 10% or more.


SEQUENCE LISTING (9 sequences)

SEQ ID NO: 1 - DNA, 15 bp, Artificial Sequence
  Chemically synthesized
acgtgtgctgtccgt

SEQ ID NO: 2 - DNA, 21 bp, Artificial Sequence
  Chemically synthesized
ggaattgtagtgactccaatg

SEQ ID NO: 3 - DNA, 19 bp, Artificial Sequence
  Chemically synthesized
ggaaggacatggtcaagat

SEQ ID NO: 4 - DNA, 19 bp, Artificial Sequence
  Chemically synthesized
cgtacgcggaatacttcga

SEQ ID NO: 5 - DNA, 84 bp, Artificial Sequence
  Chemically synthesized
tggagccatcctcagttcgagaaaggtggcggttctggcggagggtcgggcggctccgcctggagtcaccctcagtttgagaaa

SEQ ID NO: 6 - protein, 403 aa, Homo sapiens
MEPVGCCGECRGSSVDPRSTFVLSNLAEVVERVLTFLPAKALLRVACVCRLWRECVRRVLRTHRSVTWISAGLAEAGHLEGHCLVRVVAEELENVRILPHTVLYMADSETFISLEECRGHKRARKRTSMETALALEKLFPKQCQVLGIVTPGIVVTPMGSGSNRPQEIEIGESGFALLFPQIEGIKIQPFHFIKDPKNLTLERHQLTEVGLLDNPELRVVLVFGYNCCKVGASNYLQQVVSTFSDMNIILAGGQVDNLSSLTSEKNPLDIDASGVVGLSFSGHRIQSATVLLNEDVSDEKTAEAAMQRLKAANIPEHNTIGFMFACVGRGFQYYRAKGNVEADAFRKFFPSVPLFGFFGNGEIGCDRIVTGNFILRKCNEVKDDDLFHSYTTIMALIHLGSSK

SEQ ID NO: 7 - DNA, 1377 bp, Homo sapiens
gtggcgcggacgcctgctcagtgcgcgccggccgggcaaccctatgctggcgtaatcgggttcctccgagccgccgtaggactggttccggcgggctggtgaggaatggagccggtaggctgctgcggcgagtgccgcggctcctccgtagacccgcggagcaccttcgtgttgagtaacctggcggaggtggtggagcgtgtgctcaccttcctgcccgccaaggcgttgctgcgggtggcctgcgtgtgccgcttatggagggagtgtgtgcgcagagtattgcggacccatcggagcgtaacctggatctccgcaggcctggcggaggccggccacctggaggggcattgcttggttcgcgtggtagcagaggagcttgagaatgttcgcatcttaccacatacagttctttacatggctgattcagaaactttcattagtctggaagagtgtcgtggccataagagagcaaggaaaagaactagtatggaaacagcacttgcccttgagaagctattccccaaacaatgccaagtccttgggattgtgaccccaggaattgtagtgactccaatgggatcaggtagcaatcgacctcaggaaatagaaattggagaatctggttttgctttattattccctcaaattgaaggaataaaaatacaaccctttcattttattaaggatccaaagaatttaacattagaaagacatcaactcactgaagtaggtcttttagataaccctgaacttcgtgtggtccttgtctttggttataattgctgtaaggtgggagccagtaattatctgcagcaagtagtcagcactttcagtgatatgaatatcatcttggctggaggccaggtggacaacctgtcatcactgacttctgaaaagaaccctctggatattgatgcctcgggtgtggttggactgtcatttagtggacaccgaatccagagtgccactgtgctcctcaacgaggacgtcagtgatgagaagactgctgaggctgcgatgcagcgcctcaaagcggccaacattccagagcataacaccattggcttcatgtttgcatgcgttggcaggggctttcagtattacagagccaaggggaatgttgaggctgatgcatttagaaagttttttcctagtgttcccttattcggcttctttggaaatggagaaattggatgtgatcggatagtcactgggaactttatattgaggaaatgtaatgaggtaaaagatgatgatctgtttcatagctatacaacaataatggcactcatacatctggggtcatctaaataataattaaagtggctttcataatatgtaaaaaaaaaaaaaaaaaaaaaaaaaaaaaaaaaa

SEQ ID NO: 8 - protein, 28 aa, Artificial Sequence
  Chemically synthesized
WSHPQFEKGGGSGGGSGGSAWSHPQFEK

SEQ ID NO: 9 - DNA, 20 bp, Artificial Sequence
  Chemically synthesized
cgccggaaccagtcctacgg